(19) United States
(12) Patent Application Publication
(10) Pub. No.: US 2010/0004684 A1
(43) Pub. Date: Jan. 7, 2010

(54) RECLINING CHAIR SYSTEM FOR SPIRAL TRACTION

(76) Inventor: Richard J. Rogachevsky, Portland, OR (US)

Correspondence Address: SCHWEGMAN, LUNDBERG & WOESSNER, P.A., P.O. BOX 2938, MINNEAPOLIS, MN, 55402, US

(21) Appl. No.: 12/443,086

(22) PCT Filed: Sep. 27, 2007
(86) PCT No.: PCT/US07/20862

§ 371 Date: Mar. 26, 2009

Related U.S. Application Data
(60) Provisional application No. 60/848,508, filed on Sep. 27, 2006.

(51) Int. Cl. A61F 5/00; A61G 15/00

(52) U.S. Cl. 606/242; 128/845

(57) ABSTRACT

A spiral-traction device is disclosed that includes a neck and upper back frame attached designed to attached to a patient, and a portable or stationary reclining chair. The neck and upper back frame is used with the portable or stationary reclining chair. Both the neck and upper back frame and the portable or stationary reclining chair have inflatable and deflatable air chambers that can be controlled to apply spiral traction relax muscles and to release soft-tissues and decompress spinal-joint surfaces with specificity.

### CLAIM OF PRIORITY

Benefit of priority is hereby claimed to U.S. Patent Application Ser. No. 60/848,508, filed on Sep. 27, 2006, which application is herein incorporated by reference.

### BACKGROUND OF THE INVENTION

Spinal-joint distraction and soft tissue mobilization are therapeutic procedures in the field of spinal orthopedics. Spinal-joint distraction decompresses internal pressure from joint surfaces; and soft tissue mobilization releases muscle spasms and contractures, and connective tissue adhesions. Most traction or decompression devices use decompression and traction methods that distract the Spine in a straight path along the longitudinal axis of the spine.

These devices have generally been found to have limited success as compared to the results that can be obtained with comparable manual clinical procedures. Many of these devices place linear forces on the spine and are able to separate spinal joint-surfaces, but they did not relax neck muscles. Some researchers have found that traction devices do not have the ability to relax muscles. Their fundamental feature appears to be mechanical, and that is to separate the joint surfaces. Some researchers have found compression or narrowing of the joint space with application of cervical traction. This narrowing is often attributed to muscle guarding and to patients' inability to relax during traction. As a result, these types of devices have not been used widely other than for orthopedic ailments, and even then with limited success. As a result, there is a need for more effective home-care devices to distract spinal joints and release soft-tissues. There is also a need in clinical settings, for more effective clinical traction and decompression modalities that do not require constant attention from the physician, physician's assistant, or the like.

### BRIEF DESCRIPTION OF THE DRAWINGS

The invention is pointed out with particularity in the appended claims. However, a more complete understanding of the present invention may be derived by referring to the detailed description when considered in connection with the figures, wherein like reference numbers refer to similar items throughout the figures and:

FIG. 1 illustrates a schematic view of a systems 100.

FIG. 2 is a portion of a flow diagram of a method of using the system that includes the neck and upper back frame, and a reclining chair, according to an example embodiment.

FIG. 3 is a portion of a flow diagram of a method of using the system that includes the neck and upper back frame, and a reclining chair, according to an example embodiment.

FIG. 4 is a portion of a flow diagram of a method of using the system that includes the neck and upper back frame, and a reclining chair, according to an example embodiment.

FIG. 5 illustrates in perspective view an embodiment of a temporomandibular joint (TMJ) spacer, according to another example embodiment.

FIGS. 6A, 6B, and 6C are side, back and front views, respectively, of an embodiment of a neck-and-upper-back frame. FIG. 6D is a schematic top view of an embodiment of a neck frame, according to an example embodiment.

FIGS. 6E and 6F are top and side views, respectively, of an embodiment of a lateral slat, according to an example embodiment.

FIG. 6G is a perspective view of an embodiment of a front chamber, according to an example embodiment.

FIGS. 6H, 6I, and 6J are a side view in the locked position, a side view of in the unlocked position, and a rear view in the locked position, respectively, of an embodiment of a rear slat sleeve, according to an example embodiment.

FIGS. 6K and 6L are a side view and a rear view in the locked position, respectively, of an embodiment of a front slat sleeve. FIG. 6M is a side view of another embodiment of a front slat sleeve, according to an example embodiment.

FIG. 6N is a front view of an embodiment of a lateral slat sleeve, according to an example embodiment.

FIGS. 6O, 6P, and 6Q are side views of three embodiments of a rear bracket, according to an example embodiment.

FIGS. 7A and 7B are side and front views, respectively, of an embodiment of a neck-and-upper-back frame that includes a lower cervical tilt, according to an example embodiment.

FIG. 7C is a front view of an embodiment of a lower cervical tilt, according to an example embodiment.

FIGS. 8A and 8B are side and rear views, respectively, of an embodiment of a neck-and-upper-back frame that includes a adjustable chin and occipital cups, according to an example embodiment.

FIGS. 8C, 8D, and 8E are side, back, and top views, respectively of an embodiment of an adjustable occipital cup, according to an example embodiment.

FIGS. 8F, 8G, 8H, and 8I are top, bottom, detail, and cross section views of an embodiment of a rear slat, according to an example embodiment.

FIGS. 8J and 8K are side and back views respectively of an embodiment of a chin cup, according to an example embodiment.

FIGS. 8L and 8M are top and side views of an embodiment of a front slat, according to an example embodiment.

FIGS. 9A and 9B are side and front views, respectively, of an embodiment of a neck-and-upper-back frame that includes a middle cervical tilt, according to an example embodiment.

FIG. 9C is a front view of an embodiment of a middle cervical tilt, according to an example embodiment.

FIG. 10 illustrates in perspective view an embodiment of a manually operated manifold and gas bulb, according to an example embodiment.

FIG. 11 is a perspective view of a reclining chair, according to an example embodiment.

FIG. 12 is a perspective view of a reclining chair, according to another example embodiment.

FIG. 13 is a side view of a portable reclining chair 1300, according to an example embodiment.

FIG. 14 is a side view of the inflatable chambers for the thigh and leg support, according to an example embodiment.

FIG. 15 is a side view of the inflatable chamber for reclining the torso by flexing the hip, according to an example embodiment.

FIG. 16 is a front view of the portable reclining chair, according to an example embodiment.

FIG. 17 is a cross sectional view of the portable reclining chair along line 17-17 in FIG. 13, according to an example embodiment.

FIG. 18 is a cross sectional view of the portable reclining chair along line 18-18 in FIG. 13, according to an example embodiment.

FIG. 19 is a schematic of the control mechanism 1900 for a reclining chair, according to an example embodiment.

FIG. 20 is a block diagram of a computer system 2000 that executes programming for performing methods discussed herein, according to an example embodiment.

The description set out herein illustrates the various embodiments of the invention and such description is not intended to be construed as limiting in any manner.

### DETAILED DESCRIPTION

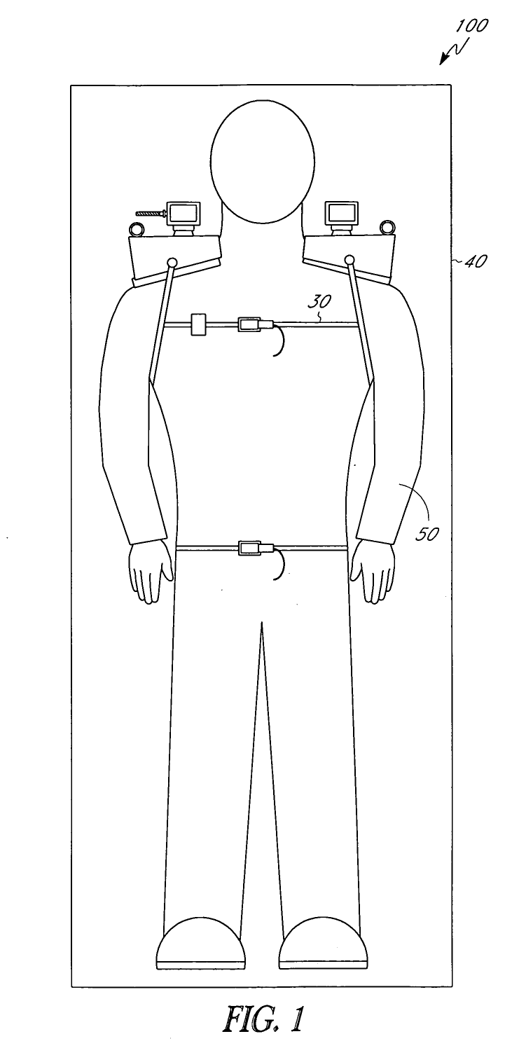

Embodiments of devices, systems, and methods exhibit at least some of the following features, which are discussed in greater detail below. The system can be thought of as including two general portions. FIG. 1 is a schematic view of a system that includes a neck and upper back frame 30 and a reclining table 40. The neck and upper back frame 30 is attached to a patient's body 50. The other portion is a reclining chair 40 that is used in conjunction with the neck and upper back frame portion 30. The reclining chair 40 can be portable or stationary. The reclining chair anchors and stabilizes the lower body, and the neck and upper back frame anchors and stabilizes the upper body. The use of both the neck and upper back frame 30 and the reclining chair 40 forms a system 100 to provide alignment to the entire spine prior to applying spiral distraction. They also provide specificity for the traction and mobilization of soft tissues. Furthermore, when medically indicated, the use of both the reclining chair, and the neck and upper back frame permits the safe use of traction and mobilization at higher velocities that approximate those utilized during spinal manipulation procedures.

The following includes a description of the various portions of the system. The neck and upper back frame portion will be described initially. After fully describing the neck and upper frame portion, the reclining chair portion will be detailed. Finally, the operation of the system will be more fully set forth.

Embodiments of the devices are portable, compact, lightweight, and easily assembled and disassembled. Accordingly, they are suitable for both clinical and home use. Embodiments of devices permit the neck to be oriented in any position (rotation, flexion, extension, side flexion prior to traction. Traction of the neck and upper back uses up to seven points of contact. The spinal traction uses spiral pathways (spiral traction). Aligning the upper-thoracic spine reduces thoracic kyphosis (hunched posture), internal rotation of the shoulders (rounded shoulders), and/or upper-thoracic rotational scoliosis. Temporo-mandibular joints (TMJs) are decompressed. The traction uses a low traction force. Described below are several embodiments of neck and upper back frames. Next described are several embodiments of reclining chairs. The neck and back frames are used in combination with the reclining chairs to form various systems.

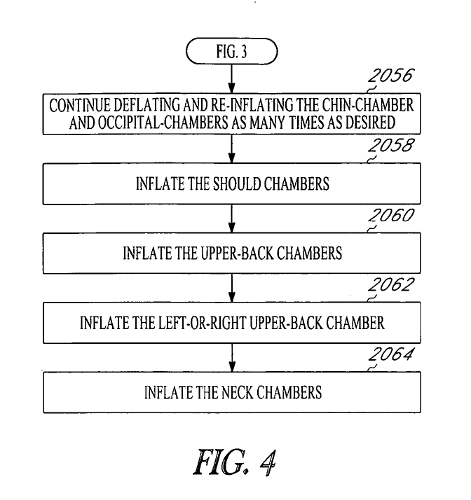

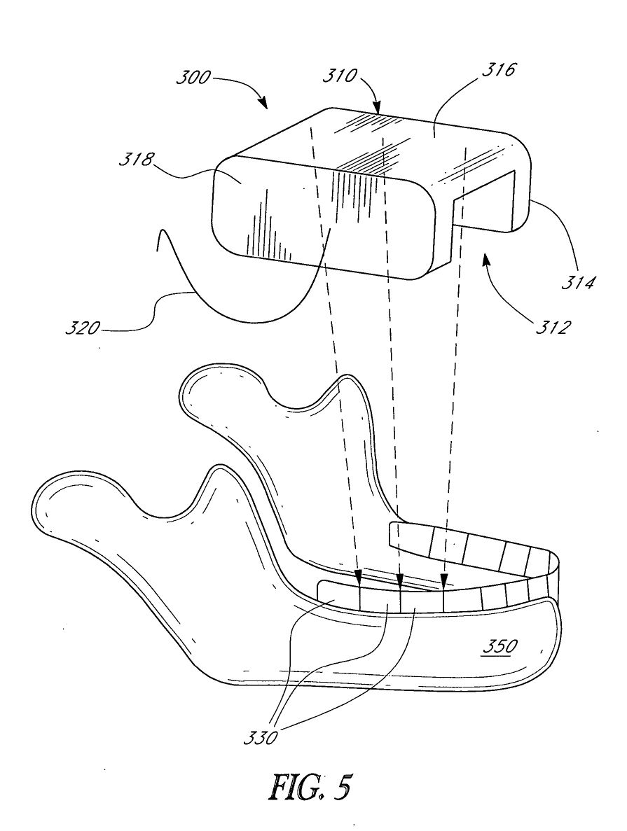

FIG. 5 illustrates an embodiment of a right temporo-mandibular joint (TMJ) or dental 300. The illustrated embodiment of the TMJ spacer comprises a body 310 and a flexible cord or leash 320. The body 310 comprises a channel 312 sized and dimensioned to cover the biting surfaces of the user's molars, and optionally, the premolars, collectively 330. In the illustrate embodiment, the covered teeth are of the lower jaw. Those skilled in the art will understand that in some embodiments, the channel 312 covers the teeth of the upper jaw, and/or both jaws. The channel 312 defines an inner wall 314, an upper wall 316, and an outer wall 318. In some embodiments, the thicknesses of the walls 314, 316, and 318 are independently from about 1 mm (0.04″) to about 4 mm (0.16″), preferably, about 1.5 mm±0.7 mm ( 1/16″± 1/32″). In some preferred embodiments, the walls 314, 316, and 318 have substantially the same thicknesses. The body 310 comprises any suitable material, for example, a polymer. Examples of suitable polymers include polyethylene, polypropylene, and the like. In preferred embodiments, the body 310 is manufactured as a single piece from a single material, thereby reducing manufacturing costs. The cord 320 is secured to the outer wall 318, and prevents swallowing of the TMJ spacer 300. A left TMJ spacer (not illustrated) is also typically used, which is substantially identical to the right TMJ spacer 300, but is disposed on the other side of the user's jaw. In some preferred embodiments, the cords 320 of the left and right TMJ spacers 300 are joined. The TMJ spacers protect the user's teeth and TMJ, as discussed below.

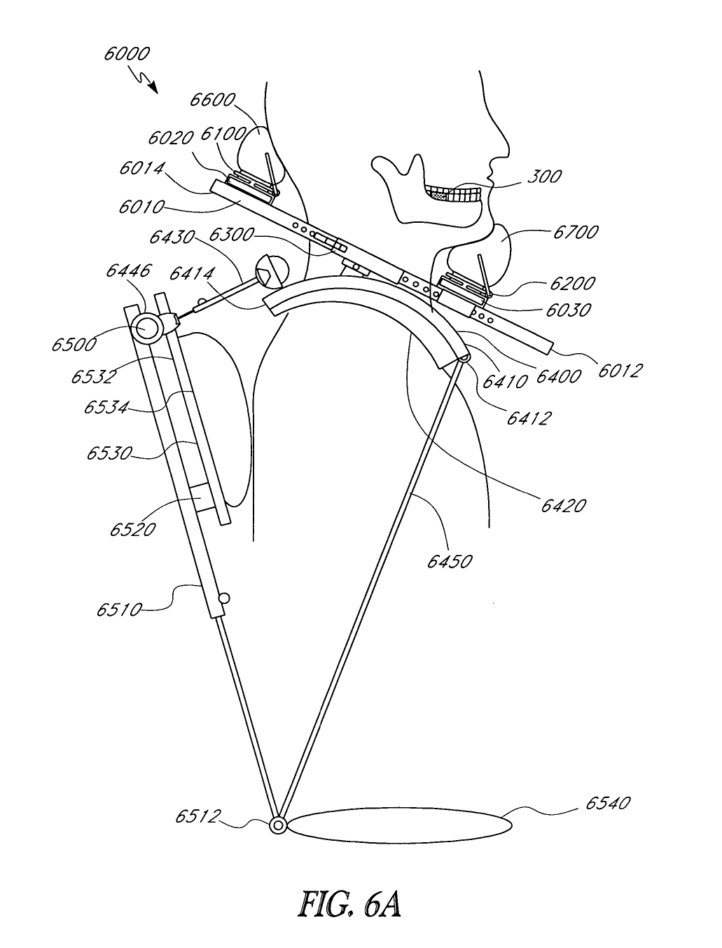

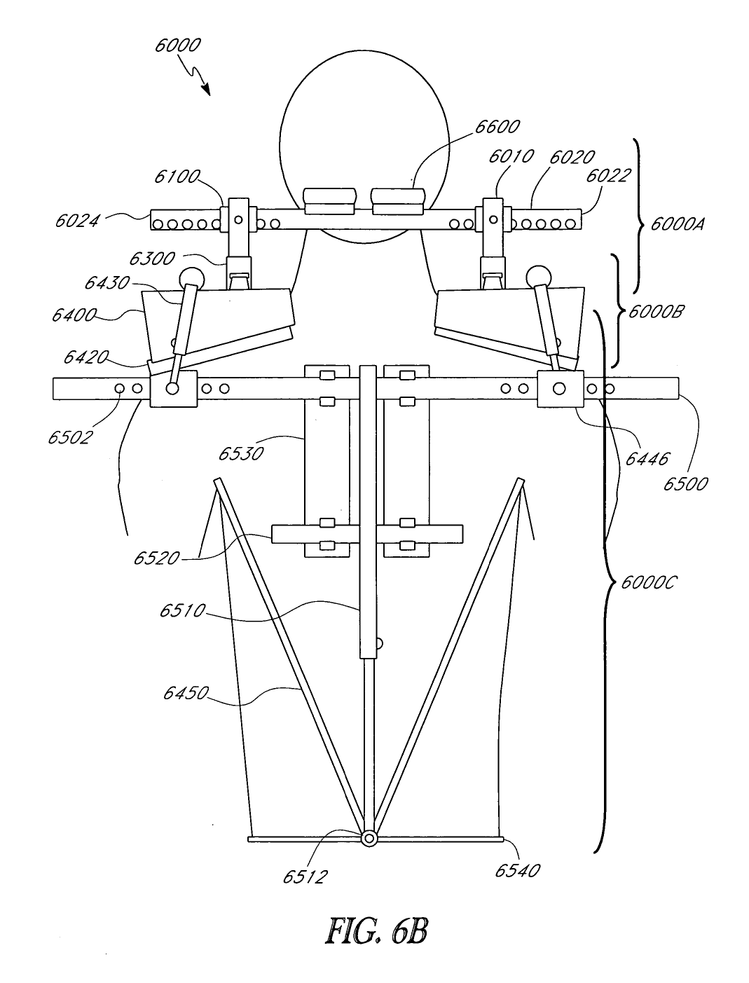

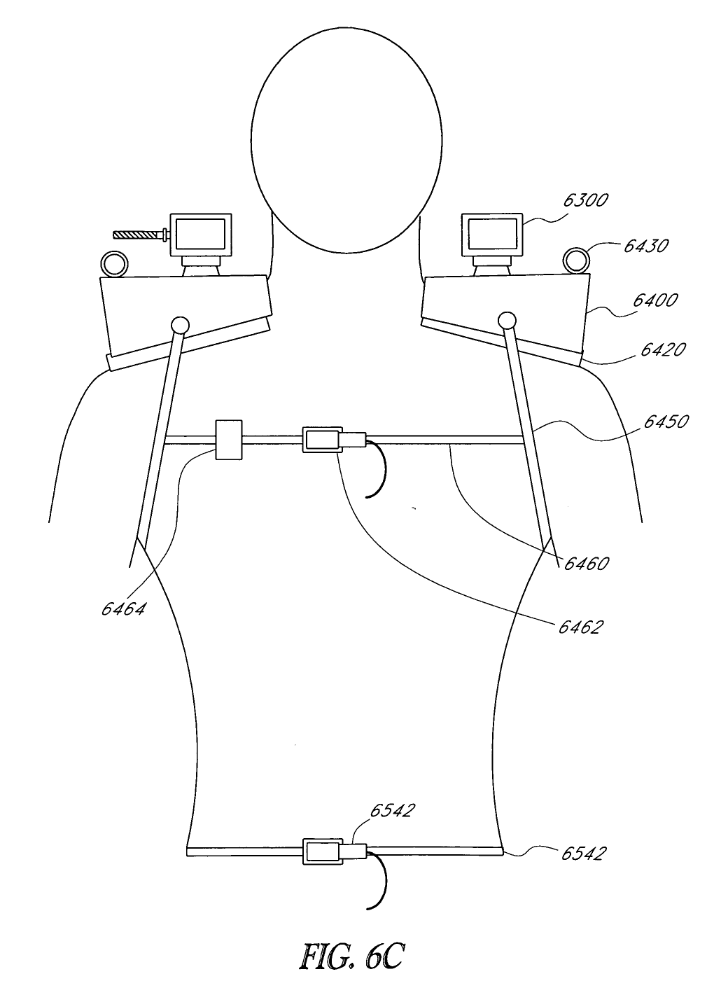

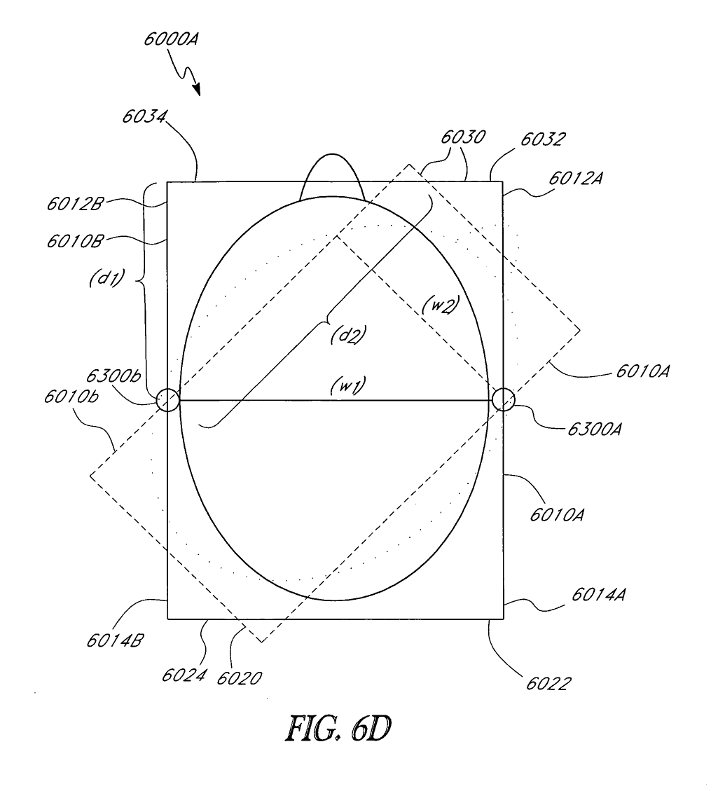

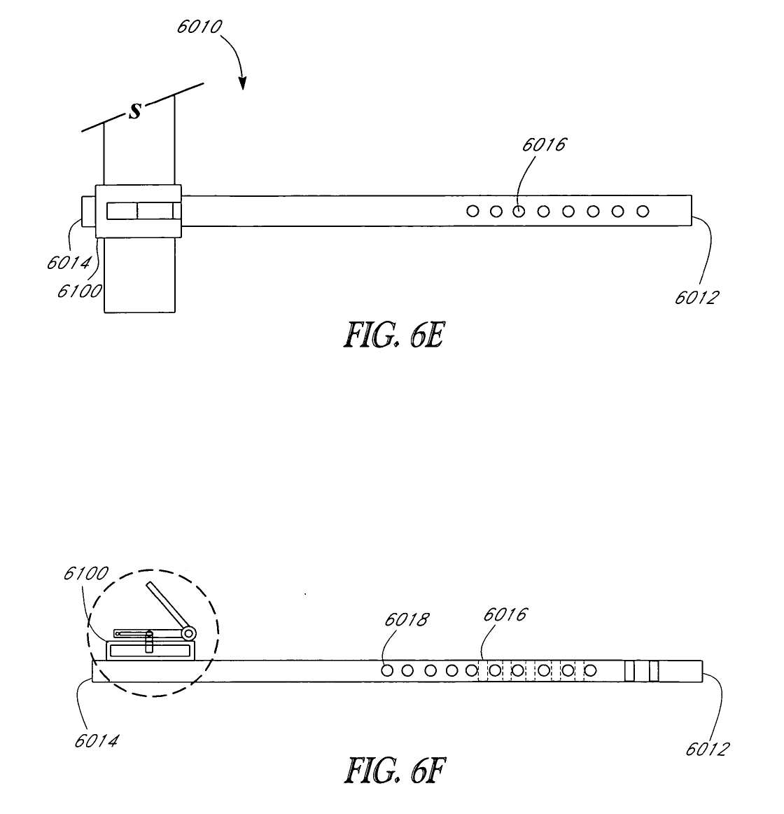

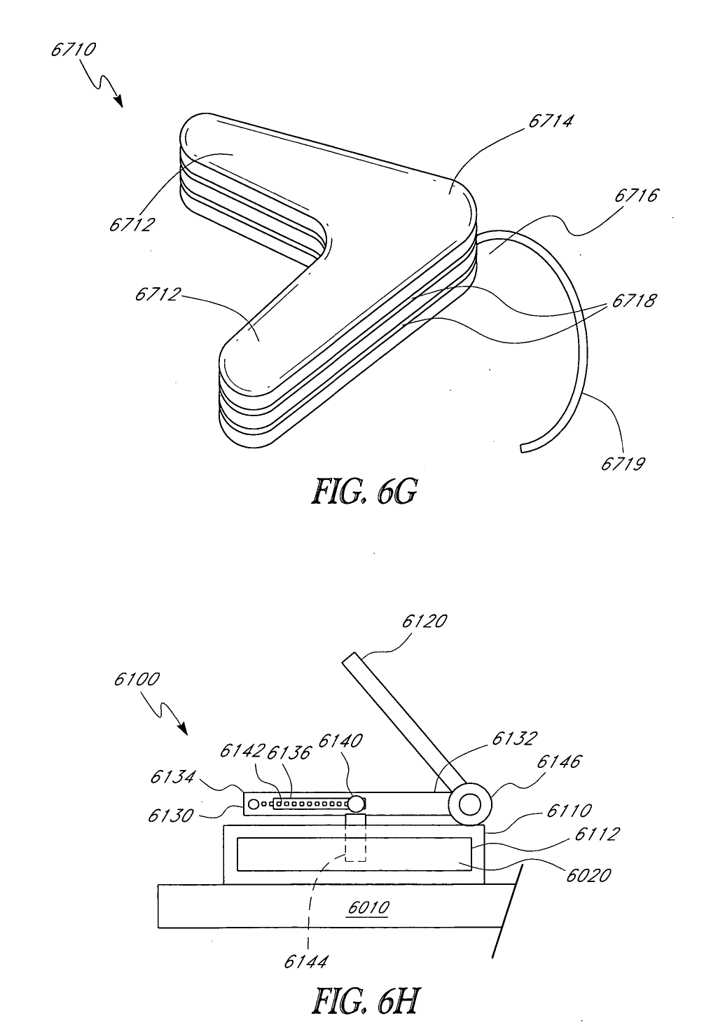

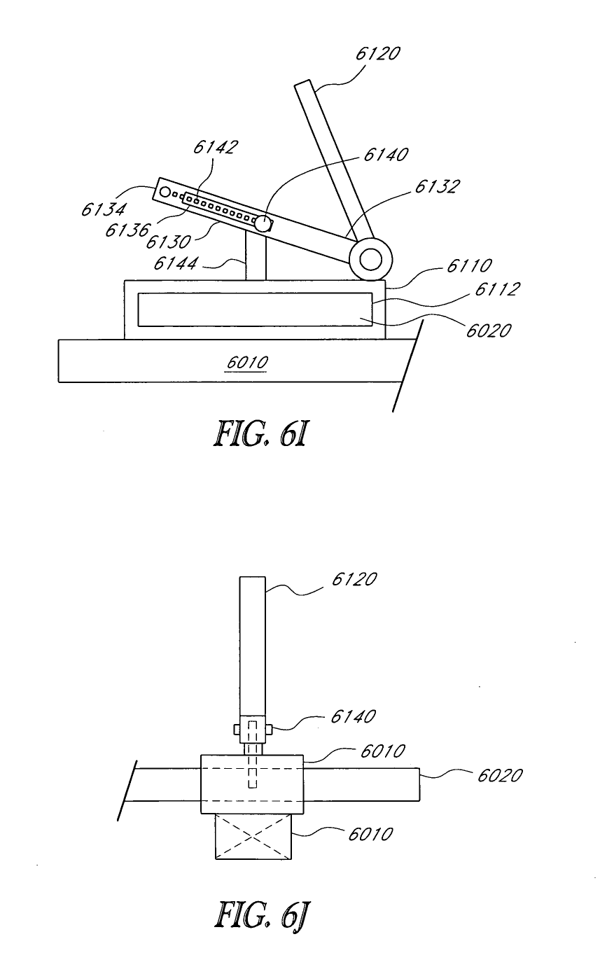

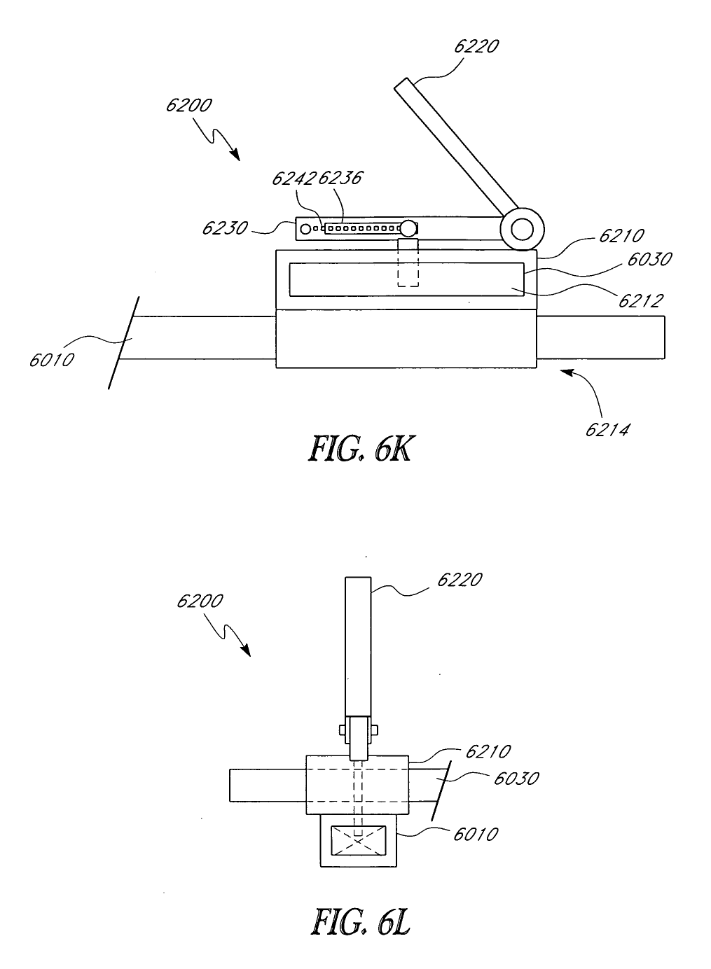

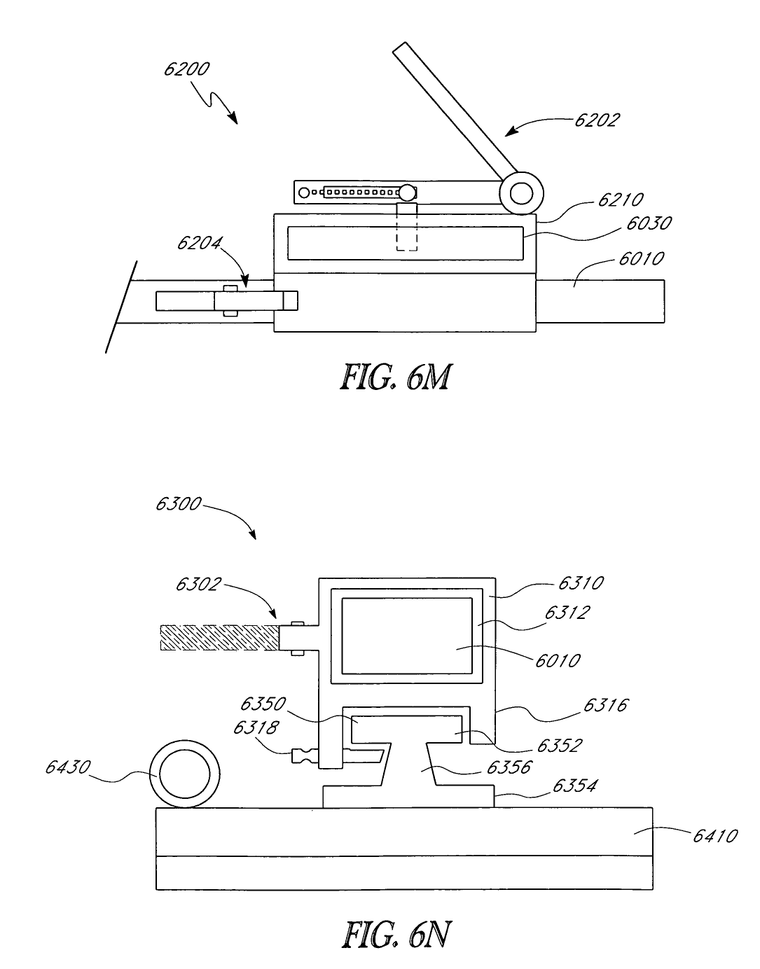

FIGS. 6A-6C are a side view, a back view, and a front view, respectively, of an embodiment of a neck-and-upper-back frame 6000 useful for applying traction to the neck and upper spine. As best seen in FIG. 6B, the device 6000 comprises a neck frame 6000a, a shoulder frame 6000b, and an upper-back frame 6000c. Portions of the neck frame 6000a are not illustrated in the front view 6C.

As best viewed in FIGS. 6A and 6B, the illustrated embodiment of the neck frame 6000a comprises a pair of lateral slats 6010, a rear slat 6020, and a front slat 6030 (not illustrated in FIG. 6B). One lateral slat 6010 is disposed on either side of the user's head. The rear slat 6020 is slidably secured to both lateral slats 6010, and is disposed behind the user's head. The front slat 6030 is slidably secured to the lateral slats 6010, and is disposed in front of the user's neck, below the chin. In a schematic top view illustrated in FIG. 6D, collectively, the right lateral slat 6010a, left lateral slat 6010b, rear slat 6020, and front slat 6030 form a rectangle. Each lateral slat 6010 comprises a first or front end 6012 and a second or back end 6014. The front end 6012 of each lateral slat is positioned in front of the user's head, and the back end 6014 is positioned behind the user's head. The rear slat 6020 comprises a first or right end 6022 and a second or left end 6024. The front slat 6030 also comprises a first or right end 6032 and a second or left end 6034. The right ends of the rear 6022 and front 6032 slats are positioned to the right of the user's head, while the left ends of the rear 6024 and front 6034 slats are positioned to the left of the user's head.

As used herein, the term “slat” refers to elongate substantially rigid structures of any suitable cross-section, and includes structures such as slats, rods, beams, tubes, rails and other structures known in the art. In some embodiments, the slats have a substantially constant cross section along the length. In other embodiment, the cross section of the slat is not constant. Slats comprise any suitable material known in the art, for example, wood, wood composites, metals, polymers, inorganic materials, and combinations thereof. In some embodiments, the slats comprise a composite, for example, a fiberglass composite, a wood composite, and/or a carbon fiber composite.

Returning to FIG. 6A, the rear slat 6020 is mounted to the lateral slats 6010 using a pair of rear slat sleeves 6100. In the illustrated embodiment, each rear slat sleeve 6100 is substantially immovably secured to a corresponding lateral slat 6010. Each rear slat sleeve 6100 permits relative lockably slidable left-right motion between the rear slat 6020 and the corresponding lateral slat 6010. In the illustrated embodiment, the rear slat sleeves 6100 maintain a substantially perpendicular relationship between the rear slat 6020 and each lateral slat 6010. Details of the construction of the rear slat sleeves 6100 are provided below.

The front slat 6030 is mounted to the lateral slats 6010 using a pair of rear slat sleeves 6200. In the illustrated embodiment, each front slat sleeve 6200 is lockably slidably mounted to a corresponding lateral slat 6010, thereby permitting front-back motion of the front slat sleeve 6200 along the corresponding lateral slat 6010. Each front slat sleeve 6200 permits relative lockably slidable left-right motion between the front slat 6030 and the corresponding lateral slat 6010. In the illustrated embodiment, the front slat sleeves 6200 maintain a substantially perpendicular relationship between the front slat 6030 and each lateral slat 6010. Details of the construction of the front slat sleeves 6200 are provided below.

FIGS. 6E and 6F illustrate top and side views respectively of an embodiment of the lateral slats 6010. Each lateral slat comprises a first or front end 6012 and a second or back end 6014. Secured towards the back end 6014 is a rear slat sleeve 6100. As illustrated in FIG. 6E, the rear slat 6020 is substantially perpendicular to the lateral slat 6010 in the rear slat sleeve 6100. A series of openings or holes 6016 extend from the first end 6012 towards the second end 6014. These openings 6016 comprise a component in the locking mechanism of an embodiment of the front slat sleeve 6200 as described below. In the illustrated embodiment, the openings 6016 extend through the lateral slat 6010 from the top to bottom. As shown in FIG. 6F, a second series of longitudinally extending holes or openings 6018 are provided on the sides of the lateral slat 6010. The openings 6018 comprise a component in an embodiment of a mechanism for locking the lateral slat 6010 in the lateral slat sleeve 6100. The openings 6018 are also useful in an embodiment of a front slat sleeve illustrated in FIG. 6M and described below. Some embodiments of the lateral slats 6010 below do not comprise openings 6016 and/or 6018, as discussed.

In the embodiment illustrated in FIGS. 6A and 6B, the rear slat 6020 and front slat 6030 are positioned above the lateral slats 6010 as viewed from the side. Those skilled in the art will understand that other arrangements are possible, for example, with both rear 6020 and front 6030 to slats positioned below the lateral slats 6010, or one of the rear 6020 or front 6030 slats above the lateral slats 6010, and the other below. In some embodiments, at least one of the rear 6020 or front 6030 slats is substantially at the same level as the lateral slats 6010, that is, not above or below the lateral slats 6010. Those skilled in the art will understand that other arrangements are possible. As on the illustrated embodiment, a rear slat sleeve 6100 is not adjustable relative to the lateral slat 6010. Those skilled in the art will understand that in other embodiments, the rear slat sleeve 6100 is adjustable relative to the lateral slat 6010, for example, forward and backward. Those skilled in the art will also understand that, in some embodiments, the front slat sleeve 6200 is not adjustable forward and backward relative to the lateral slat 6010.

The illustrated embodiment of the neck frame 6000a also comprises a right occipital cup 6600a and a left occipital cup 6600b (generally, 6600). The right occipital cup 6600a is sized and dimensioned to engage the user's right left occipital region of the head. Similarly, the left occipital cup 6600b is sized and dimensioned to engage the user's left occipital region of the head.

The occipital cups 6600 are removably mounted to the top of the rear slat 6010 and are spaced to engage a user's occipital regions of the head. The occipital cup 6600 air chambers are slightly larger than the occipital regions of the head in some embodiments. In some embodiments, at least one of the occipital cups 6600 is longitudinally adjustable along the rear slat 6010, thereby providing an adjustable distance between the two occipital cups 6600.

Also provided is a chin cup 6700 sized and dimensioned to engage a user's chin. In the illustrated embodiment, the chin cup 6700 is removably mounted to the top of and substantially at the center of the front slat 6030. Each of the occipital cups 6600 and the chin cup 6700 comprises one or more inflatable air chambers, which are configured for independent, user controlled inflation, as discussed in greater detail below.

The air chambers in the occipital cups 6600 are referred to herein as “rear chambers.” The air chamber in the chin cup 6700 is referred to herein as a “front chamber.” The air chambers comprise a flexible, substantially airtight material. In some embodiments, the air chambers comprise an elastic material. Examples of suitable materials for the air chamber are known in the art, and include polymers, natural rubber, synthetic rubber, and the like. In some embodiments, the air chamber comprises fibers and/or a fabric embedded in and/or covered with a substantially airtight material. Each of the air chambers comprises one or more inflation ports through which a gas is introduced and/or removed. One or more tubes fluidly connect the inflation ports to a source of pressurized gas, preferably through a manifold, as discussed below.

In some embodiments, at least one of the occipital cups 6600 and/or chin cup 6700 comprises a rigid and/or semi-rigid shell and/or platform to which the respective air chamber is secured. In some embodiments, the shell and/or platform is used to secure the occipital cup 6600 and/or chin cup 6700 to the rear 6020 and/or front 6030 slat, respectively. In some embodiments, the shell and/or platform shields and/or protects the air chamber, for example, by covering at least a portion of the air chamber. In some embodiments, a shell and/or platform is shaped to direct the force generated by the inflation of the air chambers. In some embodiments, the shell and/or platform comprised a lightweight and formable material, for example, a polymer, a metal, wood, a wood composite, or the like. In some embodiments, the material is a reinforced composite, for example, a fiber reinforced polymer, fiberglass, or the like. In some embodiments, one or more of the air chambers is replaceable,—for example, for providing a range of size, and/or for repair. In some embodiments, one or more of the air chambers is substantially permanently mounted to the shell and/or platform.

FIG. 6G is a perspective view of an embodiment of a front air chamber or front chamber 6710 which is mounted in the chin cup 6700. The front chamber 6710 is generally boomerang-shaped, with a pair of arms 6712 converging at an angle to form a point 6714. The sides 6716 of the air chamber comprise a plurality of corrugations 6718, which permit the front chamber 6710 to expand and contract vertically on inflation and deflation. A tube 6719 in fluid connection with the interior of the front chamber 6710 permits inflation and deflation of the front chamber. In the illustrated embodiment, the front chamber 6710 extends about half the distance from the front of the chin to the angle of the mandible. In some embodiments, the front chamber 6710 is provided in a variety of sizes to fit different users, for example, small, medium, and large sizes for adults. Some embodiments provide one or more front chambers 6710 in children's sizes.

As discussed above, some of the mechanisms in the device 6000, for example, the rear slat sleeve 6100, the front slat sleeve 6200, and the lateral slat sleeve 6300 provide releasable locking of a slat therein. Those skilled in the art will understand that any suitable locking means known in the art is useful. For example, in some embodiments disclosed herein, an opening or a hole is provided in a slat, and a plunger or pin on a slat sleeve engages the opening in the slat. The plunger is lockable using, for example, a lever. Those skilled in the art will understand that the opening or hole is a through hole in some embodiments, and a blind hole in some embodiments. This mechanism is used, for example, in embodiments of the rear slat sleeve 6100, front slat sleeve 6200, and other mechanisms described herein. Those skilled in the art will understand that other locking mechanisms known in the art are used in other embodiments. For example, in some embodiments, the locking mechanism comprises a clutch in which two adjacent pressure plates against each other are forced against each other, for example, a portion of a slat sleeve and a portion of a slat. In some embodiments, at least one of the pressure plates comprises a textured surface and/or a high friction surface. Those skilled in the art will understand that holes or openings in the slats described herein are optional in embodiments comprising a clutch.

Some embodiments described herein use a locking device known in the art referred to herein as a “push button,” which comprises a first component comprising a spring loaded button or pin biased outward, and a second component comprising at least one opening or hole sized and dimensioned to engage the pin. The mechanism is unlocked by depressing the pin clear of the second component and moving the second component relative to the first component. The mechanism is locked by moving an opening of the second component over the pin, which is biased outward, thereby engaging the opening. In some embodiments, operation of the mechanism is facilitated by rounding the top of the pin and/or chamfering the opening.

Other suitable locking means known in the art are also useful, for example, screws, detents, clips, clasps, latches, pins, pawls, notches, combinations, and the like. In some embodiments, the locking mechanism is automated, for example, using a motor, a pneumatic device, a piezoelectric device, an electromechanical device, a magnetic device, combinations thereof, and other devices known in the art.

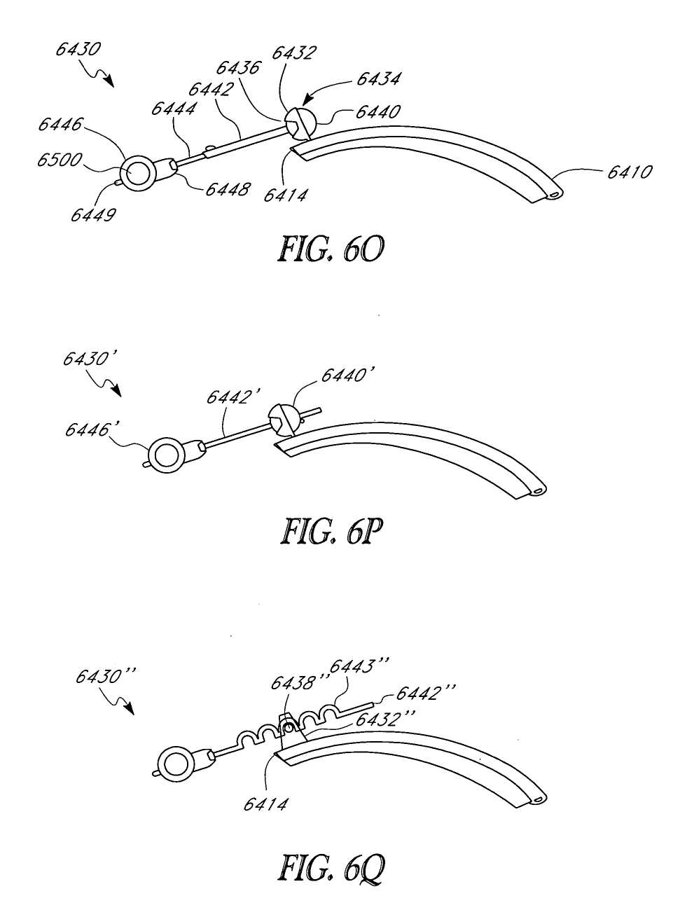

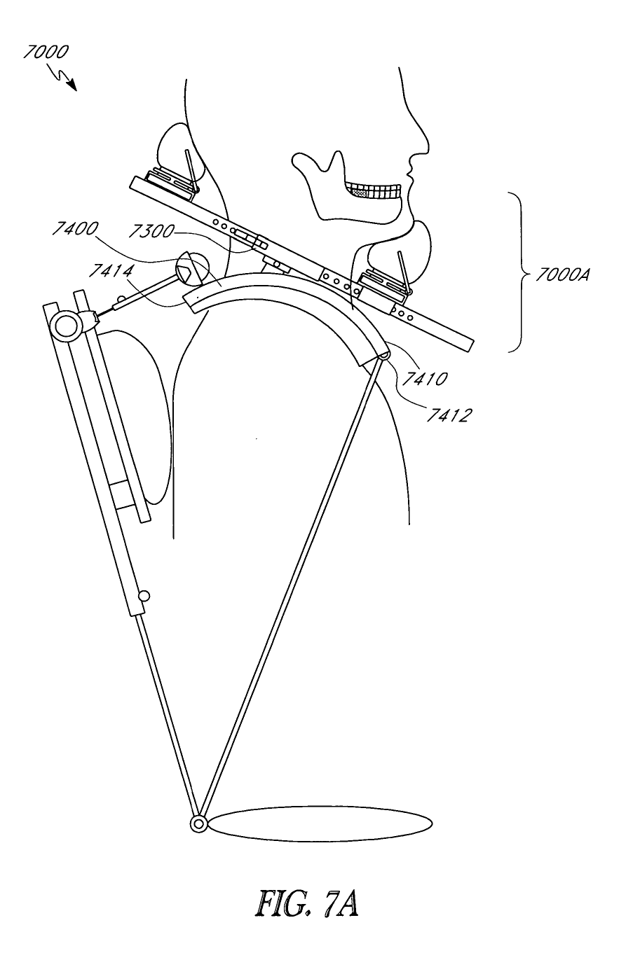

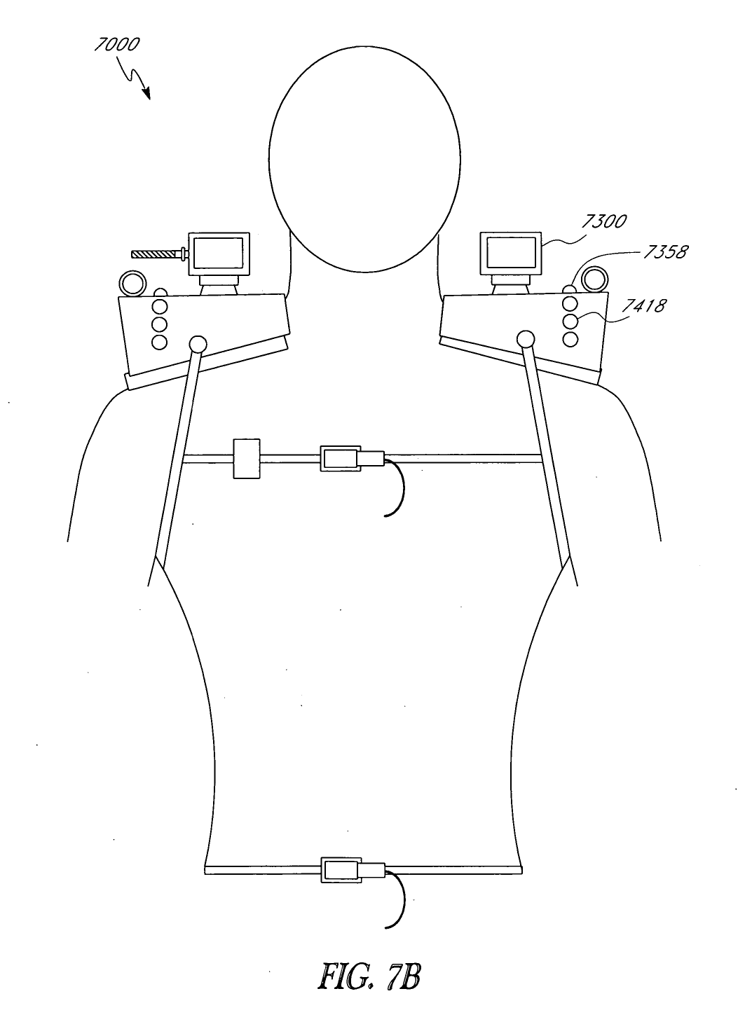

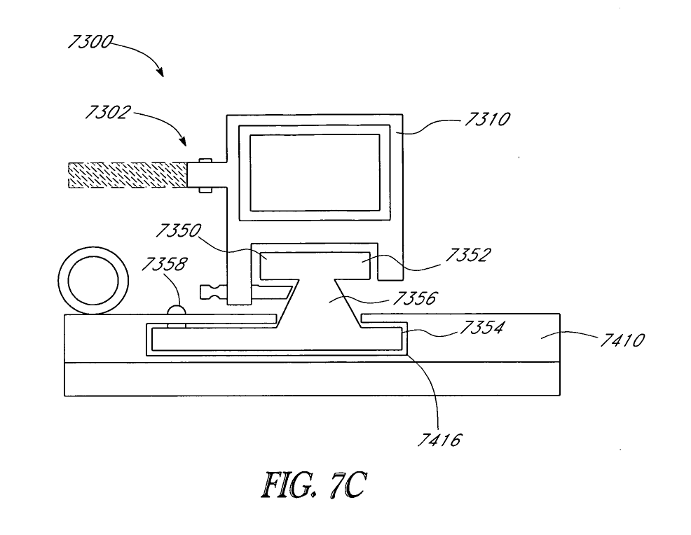

FIGS. 6H and 6I illustrate side views, and FIG. 6J illustrates a rear view of an embodiment of a rear slat sleeve 6100. As discussed above, in some embodiments, the rear slat sleeve is fixedly secured to a lateral slat 6010. The rear slat sleeves 6100 are configured to maintain the lateral slats substantially perpendicular to the rear slat 6010. Accordingly, in some embodiments, the rear slat sleeve 6100 substantially inhibits rotation between the lateral slat 6010 and the rear slat 6020. The illustrated embodiment of the rear slat sleeve 6100 comprises a body 6110, through which is formed a channel 6112 sized and dimensioned to slidably receive the rear slat 6020. The body 6110 is mounted on a lateral slat 6010. An upper lever arm 6120 secured to a lower lever arm 6130 are pivotably mounted as a single unit near an edge of the body 6110 using a hinge 6148. A slot 6136 extends from near the second end 6134 towards the first end 6132 of the lower lever arm. In the illustrated embodiment, the slot 6136 extends through the sides of and opens to the bottom of the lower lever arm 6130, resulting in a generally T-shaped cross section. A pin 6140 is slidably disposed across the crossbar of the T-shaped slot 6136, as best viewed in FIG. 7J. A spring 6142 under tension extends between the pin 6142 and the second end 6134 of the lower lever arm. Pivotably attached to the pin 6140 is a plunger 6144, which is sized and dimensioned to engage any one of a series of openings or holes 6026 in the rear slat (FIG. 6B).

FIG. 6I illustrates the rear slat sleeve 6100 in the unlocked position. Lifting the upper lever arm 6120 also lifts the lower lever arm 6130, thereby lifting the plunger 6144 from the opening 6026 in the rear slat. As the lower lever arm 6130 is lifted, the spring 6142 pulls the pin 6140 towards the second end 6134 of the lower lever arm, thereby maintaining the plunger 6144 substantially normal to the rear slat 6020 and body 6110 and preventing binding. FIG. 6J is a rear view of the rear slat sleeve 6100 in the locked position. Those skilled in the art will understand that the upper lever arm 6120 is optional in some embodiments.

FIGS. 6K and 6L illustrate an embodiment of a front slat sleeve 6200 that is substantially similar to the rear slat sleeve 6100 illustrated in FIGS. 6H-6J and described above. In the illustrated embodiment, the front slat sleeve 6200 comprises a body 6210 and a first channel 6212 formed therethrough, which sized and dimensioned to slidably receive a front slat 6030. The body 6210 also comprises a second channel 6214 sized and dimensioned to slidably receive a lateral slat 6010. The relative orientation of the first channel 6212 and the second channel 6214 constrains a perpendicular relationship between the front slat 6030 and the lateral slat 6010. The configuration of the upper lever arm 6220, lower lever arm 6230, spring 6242, slot 6236, pin 6240, and plunger 6244 are substantially as described above for the rear slat sleeve 6100. In the illustrated embodiment, the plunger 6244 simultaneously engages an opening in the front slat 6030 and an opening 6016 in the lateral slat (FIG. 6E).

FIG. 6M illustrates an embodiment of a front slat sleeve 6200, where the front slat 6030 and lateral slat 6010 are each provided with separate locking mechanisms, each of which is substantially similar to the locking mechanisms described above for the rear slat sleeve 6100. A first locking mechanism 6202 engages an opening in the front slat 6030. A second locking mechanism 6204 engages an opening 6018 in the lateral slat 6010 (FIG. 6F).

Returning to FIGS. 6A-6C, the shoulder frame 6000b comprises a pair of lateral slat sleeves 6300 mounted to corresponding shoulder pads 6400. The lateral slats 6010 are mounted to the lateral slat sleeves 6300, thereby operatively joining the neck frame 6000a to the shoulder frame 6000b. Each shoulder pad 6400 comprises a body 6410 comprising a relatively rigid, strong, and lightweight material, known in the art for example, wood composites, polymer composite, fiberglass, metal, or the like. In the illustrated embodiment, the body 6410 is sized and dimensioned to conform to a shoulder. The body comprises a front end 6412 and a back end 6414. To the underside of the body 6410 is secured an inflatable shoulder chamber 6420. The shoulder chamber 6420 has an arched shape sized and dimensioned to conform to a user's shoulder, and in the illustrated embodiment, extends from the sternum to the acromion process of the scapula, and from the second or third anterior intercostal space to the top of the scapula. Suitable materials for the shoulder chamber 6420 are discussed above and below.

To the back end 6414 each shoulder pad is mounted a rear bracket 6430 extending backwards. Details of the rear bracket are provided in greater detail below. A shoulder strap 6450 mounted to the front end 6412 of the shoulder pad, for example, to an eyelet. A chest strap 6460 extends across a user's chest between the shoulder straps 6450. In the illustrated embodiment, the chest strap 6460 comprises an adjustable clasp or buckle 6462. In the illustrated embodiment, the chest strap 6460 further comprises a clip 6464 for mounting the control manifold, which is discussed in greater detail below.

FIG. 6N illustrates a front view of an embodiment of a lateral slat sleeve 6300. In the illustrated embodiment, the lateral slat sleeve 6300 comprises a body 1110, through which a channel 6312 is formed. The channel 6312 is sized and dimensioned to slidably receive a lateral slat 6010 therethrough. A lever-and-plunger-type locking mechanism 6302 of the type described above is provided on the body 6310. The locking mechanism 6302 releasably engages an opening 6018 in the lateral slat (FIG. 6F), thereby controlling the sliding of the lateral slat 6010 in the channel 6312. A bushing 6316 is formed on the lower portion of the body 1110. The bushing 6316 is sized and dimensioned to accept and rotate on an enlarged head 6352 of a tilting lever 6350. In the illustrated embodiment, the head 6352 is substantially cylindrical. The head 6352 is formed on tilting lever 6350, which also comprises a tape 6354 and a shank 6356 extending between the tape 6354 and head 6352. The shank 6356 has a smaller diameter than the head 6352. Also provided are a one or more retaining pins 6358 which retain the bushing 6316 on the head 6352 of the tilting lever. In some embodiments, the retaining pin or pins 6358 are removable to permit disassembly. In the illustrated embodiment, the base 6354 of the tilting lever is substantially fixedly secured to the top of the shoulder pad body 6410, and oriented to provide a forward tilt to the neck frame 6000a, as illustrated in FIG. 6A.

FIGS. 60-6Q illustrate side views of three different embodiments of a rear bracket 6430, a pair of which help to secure the shoulder frame 6000b to the upper-back frame 6000c. Referring to FIG. 60, the rear bracket 6430 comprises a cup 6432 with a hemispherical interior mounted to the back end 6414 of the body of the shoulder pad. The cup 6432 comprises a first opening 6434 opening towards the front. A rear opening 6436 is provided opposite the front opening 6434. Mounted in the cup 6432 is a ball 6440 sized and dimensioned to rotate and pivot therein. A front arm 6442 extends backward from the ball 6440 through the rear opening 6436 of the cup. In the illustrated embodiment, a rear arm 6444 telescopically extends from the front arm 6442. The front 6442 and rear 6444 arms are relatively lockable, thereby providing an adjustable overall length. A sleeve 6446 is mounted to the end of the rear arm 6444 using a ball and socket joint 6448. The sleeve is sized and dimensioned to slidably receive an upper rod 6500 described below. Also illustrated in FIG. 6O is an optional push button locking mechanism 6449 that engages corresponding openings 6502 formed on the upper rod.

FIG. 6P illustrates another embodiment of a rear bracket 6430′ which is similar to the embodiment illustrated in FIG. 60. The illustrated embodiment comprises only a single arm 6442′ extending between the ball 6440′ and the sleeve 6446′. In the illustrated embodiment, the arm 6442′ extends through the ball 6440′, which comprises a locking mechanism of any type known in the art, for example, a push button lock.

FIG. 6Q illustrates another embodiment of a rear bracket 6430″. In this embodiment, a bracket 6432″ is mounted towards the rear 6414 of the shoulder pad. A pin 6438″ extends laterally from the bracket 6432″. An arm 6442″ is equipped with a plurality of hooks 6443″, which are sized and dimensioned to engage the pin 6438″. Applying tension to the structure locks the selected hook 6443″ to the pin 6438″.

As text viewed in FIG. 6B, the upper-back frame 6000c is operatively connected with the shoulder frame 6000b through the rear bracket 6430 and the shoulder straps 6450. The upper-back frame 6000c comprises an upper rod 6500 slidably mounted to the rear sleeves 6446 brackets. A telescoping vertical rod 6510 is mounted to about the center of the upper rod, for example, using a clip, and extends downwards therefrom. A lower rod 6520 is mounted to the vertical rod 6510 below the upper rod 6500 for example, using clip. The upper 6500 and lower rods 6520 are substantially perpendicular to the vertical rod 6510. Mounted to the upper 6500 and lower 6520 rods, and flanking the vertical rod 6520, is a pair of back plates 6530. Each back plate 6530 comprises a body 6532, which comprises a relatively rigid, strong, and lightweight material, an independently inflatable upper-back chamber 6534. A mount point 6512, for example, an eyelet, is provided at bottom of the vertical rod 6510 to which the shoulder straps 6450 are secured. In the illustrated embodiment, a hip belt 6540 is also mounted to the mount point 6512. As best viewed in FIG. 6C, the hip belt 6540 comprises an adjustable clasp or buckle 6542.

FIG. 6D schematically illustrates a top view illustrating a user's head and the positions of the right 6010a and left 6010b lateral slats, the rear slat 6020, and the front slat 6030. Left and right lateral slat sleeves 6300 are indicated by open circles. FIG. 7D illustrates the swiveling and alignment mechanism of the neck frame 6000a which permits rotational and translational positioning of the user's head. Because the lateral slat sleeves 6300 are positioned on the shoulder pads 6400 (not illustrated in this figure), the distance w.sub.1 is constant. Accordingly, and as will become apparent, in some embodiments, no locking mechanism is needed to control the rotational degree of freedom of the lateral slat sleeves 6300. Illustrated in solid is a user's head and neck frame 6000a with the user facing forward.

Illustrated in phantom is a user's head and neck frame 6000a after a rotation to the right. As shown in the solid lines, the distance between the left lateral slat sleeve 6300a and the front slat 6030 is indicated by d.sub.1 when the user's head is facing straight ahead. On rotating the head to the right, the distance between the left lateral slat sleeve 6300a and the front slat 6030 changes to d.sub.2 as the left lateral slat 6010b slides forward in the left lateral slat sleeve 6300b. Concomitantly, the right lateral slat 6010a slides backward in the right lateral slat sleeve 6300a to the position indicated because the rear slat sleeves and the front slat sleeves permit sliding of the rear slat 6020 and front slat 6030, respectively, but do not permit rotation. Accordingly, the neck frame 6000a is constrained to remain substantially rectangular. Consequently, on rotating the user's head to the right, as indicated in FIG. 6D, the original width w.sub.1 between the two rear slat sleeves or the two front slat sleeves changes to the width w.sub.2. As discussed above, the rear slat sleeves and front slat sleeves are lockable. Accordingly, when the positions of the rear slat 6020 and front slat 6030 are locked relative to the lateral slats 6010, the resulting rectangle is also locked. If at least one of the lateral slats 6010 were not lockable in a lateral slat sleeve 6300, the rectangle could slide forward and/or backward in the lateral slat sleeves 6300. Providing a locking mechanism for the sliding motion on either the right 6300a or left 6300b lateral slat sleeves, however, is sufficient to prevent the neck frame 6000a from moving. Accordingly, in some embodiments, a locking mechanism for the lateral slat 6010 is provided on only one of the right 6300a or left 6300b lateral slat sleeves. In other embodiments, locking mechanisms are provided on both.

It should be understood that different arrangements for the neck frame 6000a are used in other embodiments, for example, with a different geometry, and/or with more or fewer slats. In some embodiments, the slats form a different shape, for example, a pentagon, hexagon, or another polygon. In some embodiments, at least one of the slats is not generally straight, for example, curved, or a horseshoe shape.

The neck frame 6000a also provides translational alignment of the head and neck. Front-back alignment is accomplished by sliding the lateral slats 6010 forward or backward in the lateral slat sleeves 6300, and locking at least one of the lateral slat sleeves. Side-to-side alignment is provided by sliding the back 6020 and front 6030 slats in concert in the back 6100 and front 6200 slat sleeves, and locking the back 6100 and front slat sleeves 6200. FIGS. 7A and 7B illustrate side and front views, respectively, of another embodiment of a neck-and-upper-back frame 7000. In the illustrated embodiment, the neck frame 7000a swivels on the lateral slat sleeves 7300 and has an adjustable lower cervical tilt using a tilting mechanism described below.

FIG. 7C illustrates a front view of a lateral slat sleeve 7300. The body 7310 and locking mechanism 7302 are substantially similar to the embodiment of the lateral slat sleeve 6300 described above. The tilting lever 7350 is also similar, comprising an enlarged head 7352, shank 7356, and base 7354. The base 7354 is modified compared with the base in the embodiment 6300, however. In the illustrated embodiment, the base 7354 is sized and dimensioned to be slidably received in a channel 7416 formed in the body 7410 of each shoulder pad 7400. In the illustrated embodiment, a push button locking mechanism 7358 is also provided to permit user control of the tilt. The push button 7358 engages suitable holes or openings 7418 (FIG. 7B) provided on the body 7410 of the shoulder pad. A line of openings 7418 extends substantially in parallel with the channel 7416. The channel 7416 extends from the front end 7412 of the frame towards the back end 7414.

In use, the tilting lever 7350 (and lateral slat sleeve 7300) is unlocked by depressing the push button lock 7350. The forward-backward position of the lateral slat sleeve 7300 is adjusted by sliding the base 7354 in the channel 7416, and the position locked when the push button lock 7350 engages the desired opening 7418.

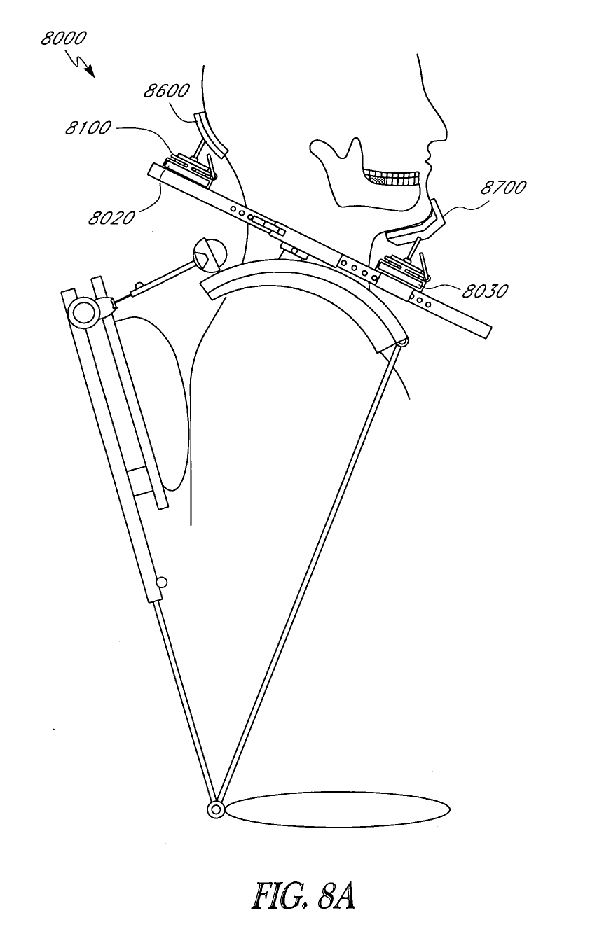

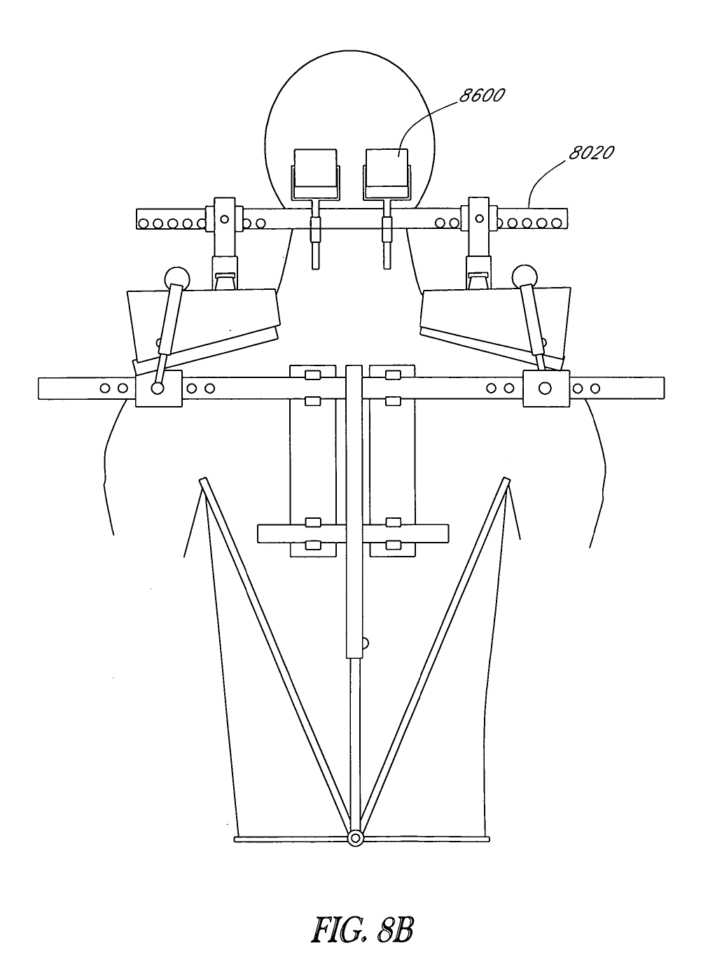

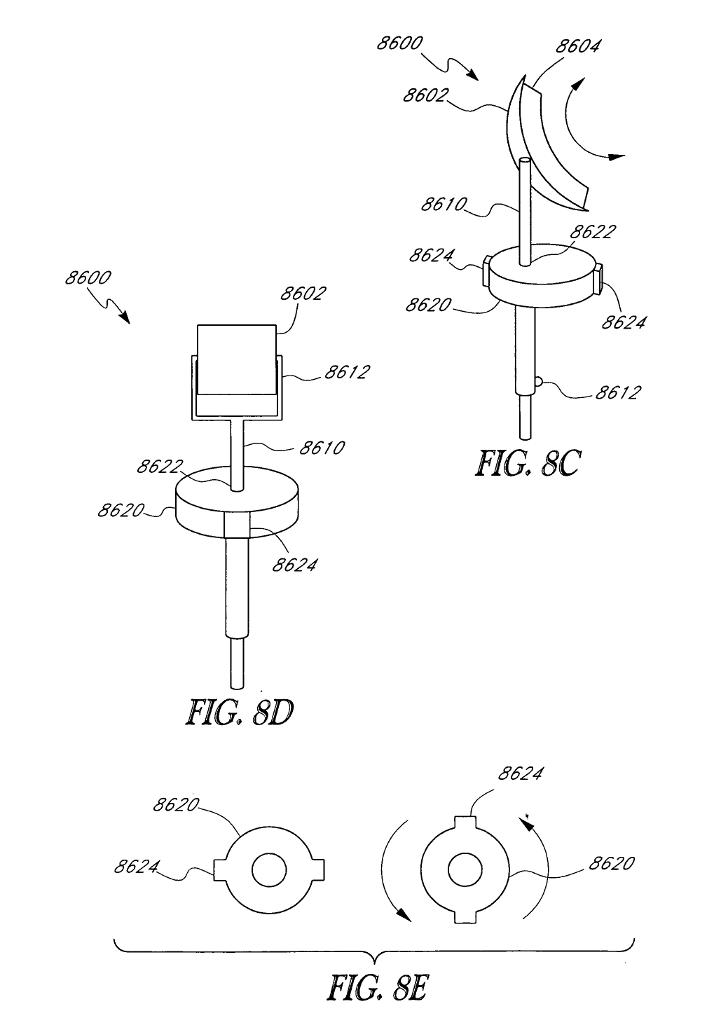

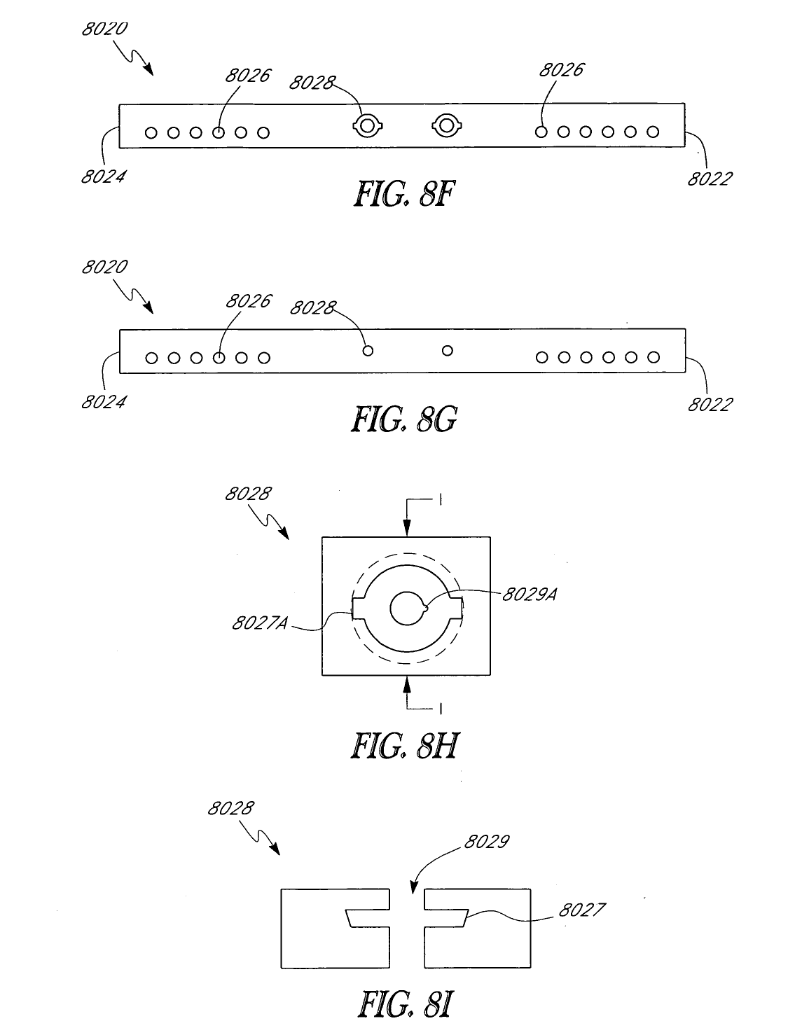

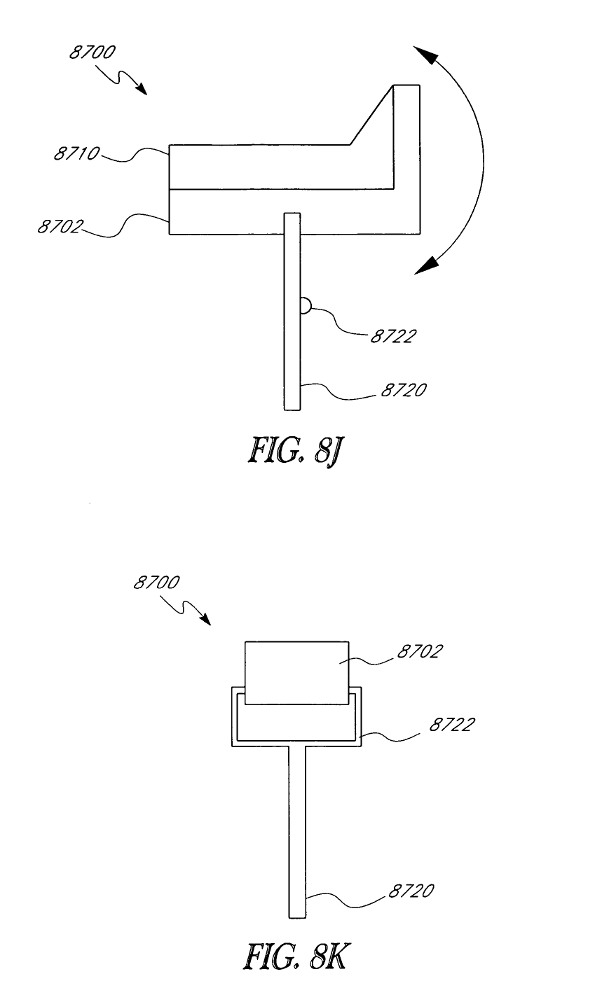

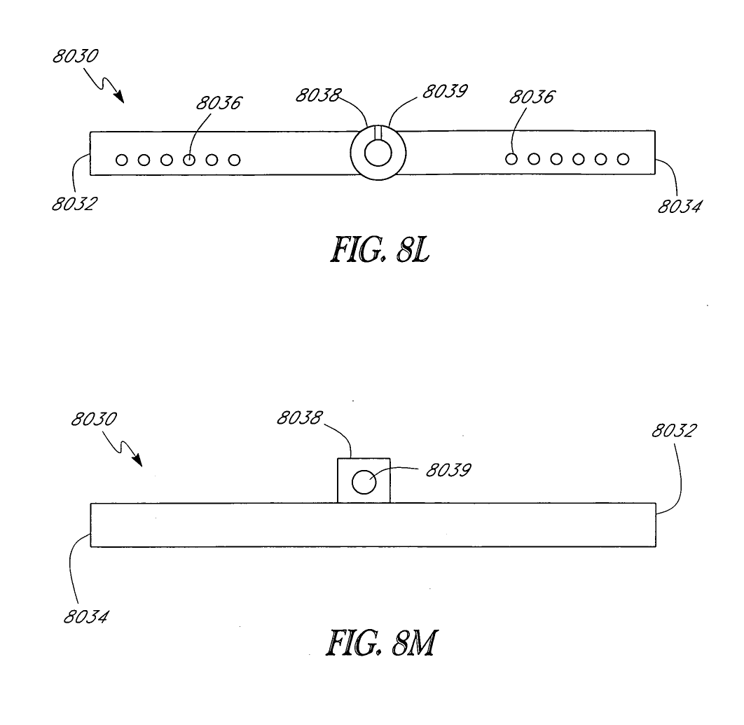

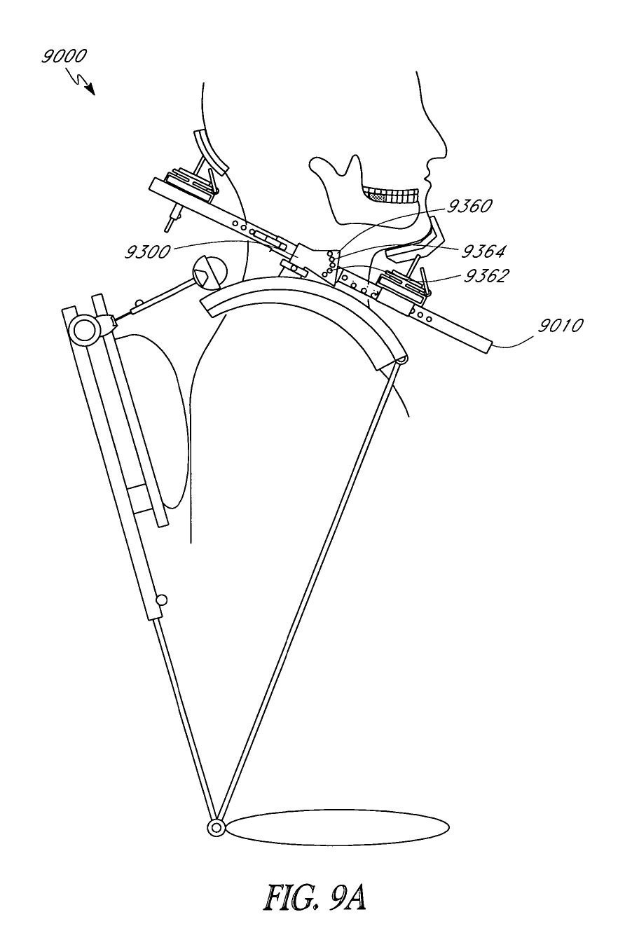

FIGS. 8A and 8B illustrate in side view and back view an embodiment of a neck-and-upper-back frame 8000, which is similar to the embodiment 7000 illustrated and described above, and further comprises adjustable occipital cups and an adjustable chin cup.

In the illustrated embodiment, the height of each occipital cups 8600 is user adjustable. Each occipital cup 8600 is also equipped with self-adjusting swivel and tilt. Similarly, the height of the chin cup 8700 is user adjustable, and equipped with self-adjusting forward and backward tilt. The user controlled and self-adjustment mechanisms are of any suitable type known in the art.

FIGS. 8C and 8D are side and back views, respectively, of an embodiment of an adjustable occipital cup 8600. The occipital cup 8600 comprises a body 8602 in which an inflatable rear chamber 8604 is disposed. As best seen in FIG. 8D, the body 8602 is pivotably mounted to a post 8610 using a pair of pivot arms 8612, thereby providing sagittal tilt as indicated by the arrow in FIG. 8C. The post is, in turn, mounted to a sleeve 8620 comprising an opening 8622 through which the post 8610 is slidable. Height adjustment is provided in the illustrated embodiment using a push button 8612 mounted to the post 8610, which engages corresponding openings on the sleeve 8620. The sleeve 8620 also comprise a pair of tabs 8624, which are sized and dimensioned to engage a radial groove formed in a rear slat as described below. FIG. 8E illustrates top views with arrows illustrating the rotational adjustment of the sleeve 8620.

FIGS. 8F and 8G are a top view and a bottom view of an embodiment of a rear slat 8020 used in conjunction with the occipital cups 8600. The rear slat 8020 comprises a first or right end 8022 and a second or left end 8024. A series of holes or openings 8026 extends toward the center from either end of the rear slat 8020. The openings 8026 are used in combination with the rear slat sleeve 8000 for locking the rear slat 8020. Near the center of the rear slat 8020 is provided a pair of openings 8028 sized and dimensioned for mounting the sleeves 8620 occipital cups. FIG. 8H illustrates a close up top view of an opening 8028. FIG. 8I is a cross-section of the opening 8028 taken through section I-I in FIG. 8H. As illustrated in FIG. 8I, the opening 8028 comprises a hole 8029 extending through the rear slat 8020, and a radial groove 8027. As shown in FIG. 8H, a pair of notches 8027a are provided, which are sized and dimensioned provide access to the radial groove 8027 by the tabs 8624 of the sleeve of the occipital cup 8600. Rotating the tabs 8624 in the radial groove 8027 captures them therein. This arrangement permits the tabs 8624 to rotate freely in the radial groove 8027. In the illustrated embodiment, the hole 8029 comprises a longitudinal groove 8029a, which provides clearance for the push button 8612 on the post of the occipital cup 8600.

FIGS. 8J and 8K illustrates an embodiment of an adjustable chin cup 8700 which comprises a body 8702 which is in the illustrated embodiment is generally L-shaped. Disposed within the L of the body 8702 is the front air chamber 8710, which is similar to the air chamber 6710 described above. The body 8702 of the chin cup is mounted on a pair of pivot arms 8722, which are in turn mounted to a post 8720, thereby providing a self-adjusting sagittal tilt as indicated by the arrows in FIG. 8J. The post 8720 is sized and dimensioned to be received in a sleeve mounted on a front slat 8030, as discussed below. Height adjustment is provided through a push button 8722 that cooperates with a corresponding opening in the sleeve, described below. Other embodiments use other adjustment means are known in the art.

FIGS. 8K and 8L are top and front views respectively of an embodiment of the front slat 8030 used with the chin cup 8700. The front slat comprises a first or right end 8032 and a second or left end 8034. A plurality of openings or holes 8036 extend from either end towards the center, which are used in conjunction with the front slat sleeve 8200 to lock the front slat 8030 in position. A sleeve 8038, which is sized and dimensioned to receive the corresponding post 8720 on the chin cup, is mounted at about the center of the top of the front slat 8030. An opening 8039 is provided at the front of the sleeve 8030 that engages the corresponding push button 8722 on the post of the adjustable chin cup, which provides height adjustment.

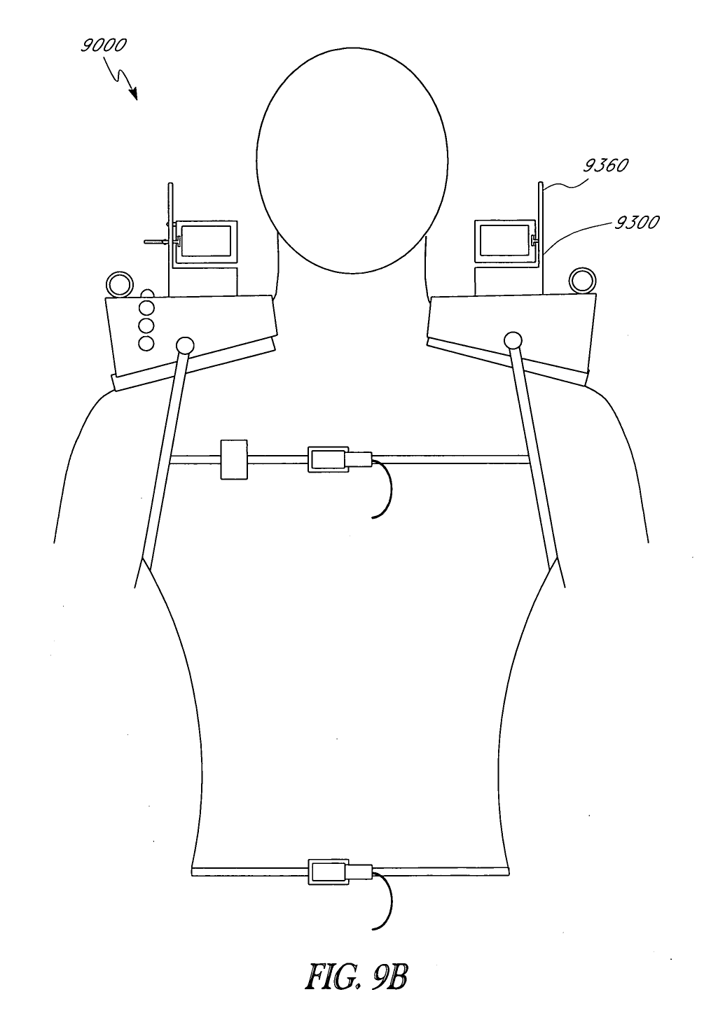

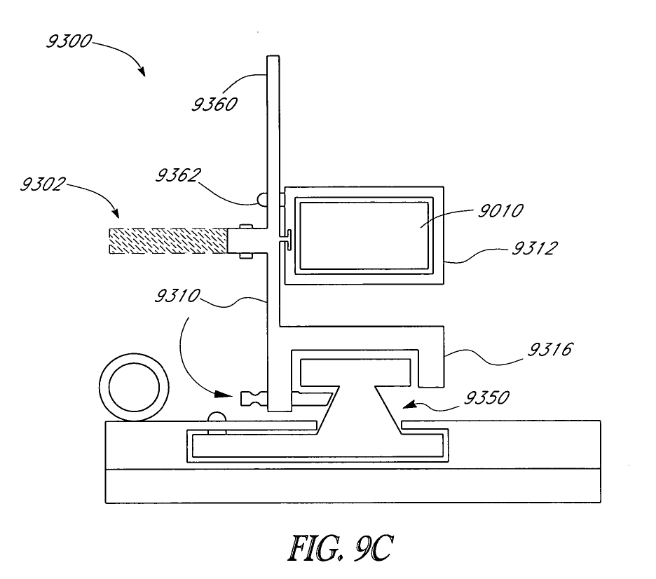

Another embodiment of the neck-and-upper-back frame 9000 illustrated in FIGS. 9A and 9B in side view and front view, respectively. The embodiment 9000 is similar to the embodiment 8000 described above, with the addition of a middle cervical tilt feature described below. As best seen in FIG. 9A, the lateral slat sleeve 9300 includes a middle tilt locking mechanism, which permits the user to tilt the lateral slats 9010 upwards and downwards.

Those skilled in the art will understand that other embodiments provide adjustability of the either of the occipital cups 8600 and/or chin cup 8700 using different means, configurations, or structures know in the art, for example, ball joints, hinges, screws, racks-and-pinions, gears, resilient structural and/or support members, fluid-filled pistons, combinations thereof, and the like. Furthermore, those skilled in the art will understand that either of the occipital cups 8600 and/or chin cup 8700 has a different shape and/or dimensions in other embodiments.

FIG. 9C illustrates a front view of a lateral slat sleeve 9300 implementing a middle cervical tilt feature. The lateral slat sleeve 9300 comprises a body 9310 at the lower portion of which is formed a bushing 9316, which is substantially similar to the bushings described above in the embodiments of the lateral slat sleeves 6300 and 7300. The arrangement of the tilting lever 6350 is similar to the tilting lever of the embodiment of the lateral slat sleeve 7300 described above.

The body 9310 comprises a pivot and locking plate 9360 extending from a side of the bushing 9316, such that the faces of the plate 9360 face left and right, as illustrated in FIG. 9A. A channel 9312 is pivotably mounted to the plate 9360 to provide a middle cervical tilt. The channel 9312 is sized and dimensioned to slidably receive a lateral slat 9010 therethrough. A lever and plunger locking mechanism 9302 is provided for locking the sliding motion of the lateral slat 9010 in the channel 9312. The channel 9312 is equipped with a push button locking mechanism 9362 that cooperates with a plurality of holes or openings 9364 formed on the plate to lock the up and down pivoting motion of the middle cervical tilt. Those skilled in the art will understand that the openings 6364 are disposed at a substantially constant radius from the middle-tilt pivot point.

Each of the neck-and-upper-back frames 6000, 7000, 8000, and/or 9000 comprises one or more features that are independently applicable and/or combinable in other embodiments. For example, the embodiment 6000 includes a swiveling neck frame that permits rotational and translational alignment of the head. The swiveling neck frame feature is present on each of the disclosed embodiments. The embodiment 7000 includes a lower cervical tilt feature, implemented using adjustable tilt levers that are longitudinally adjustable on the shoulder pads. The embodiment 8000 includes the lower cervical tilt feature, as well as adjustable occipital and chin cups. The embodiment 9000 adds a middle cervical tilt feature to the embodiment 8000 implemented in the lateral slat sleeves. Each of these embodiments also includes other features. Those skilled in the art will understand that some embodiments implement the features of the neck-and-upper-back frames 6000, 7000, 8000, and/or 9000, in different combinations.

Each of the disclosed neck-and-upper-back frames comprises seven air chambers. The neck frame comprises a left and a right rear chamber, each engaging the corresponding left and right occipital regions of the head (also referred to herein as “occipital processes,” “occipital protuberances,” or “occipital regions”), and a front chamber disposed under the user's chin. The shoulder frame comprises a left and a right shoulder chamber. The upper-back frame comprises a left and a right upper-back chamber. Each of the air chambers comprises a suitable flexible and gas tight material known in the art, for example, a polymer, natural rubber, synthetic rubber, combinations thereof, and the like. In some embodiments, the material is a composite, for example, fibers and/or fabric impregnated with and/or covered with a flexible and gas tight material. In some embodiments, the material is elastomeric. Suitable materials for air chambers are discussed in greater detail above. Each air chamber includes one or more inflation ports through which the air chamber is inflated and/or deflated. Fluidly connecting an inflation port with a source of pressurized gas causes an air chamber to inflate, and fluidly opening an inflation port to ambient or sub-ambient pressure causes the air chamber to deflate.

Those skilled in the art will understand that in some embodiments, pressurized gas is supplied to one or more of the inflation ports of the air chambers through tubing fluidly connected to one or more manifolds of any suitable type known in the art. The tubing is of any suitable type known in the art, for example, rubber, vinyl, silicone, plastic, metal, combinations thereof, and the like. In some embodiments, the deflation of one or more of the air chambers is also implemented using one or more manifolds. In some preferred embodiments, the inflation and deflation all of the air chambers are controlled using a manifold. The manifold is user controlled, automated, or a combination thereof. In some preferred embodiments, the manifold is automated, for example, controlled by a computer, microprocessor, embedded processor, or the like. In some embodiments, a user generated pressurized gas, for example, a hand bulb, hand pump, or foot pump, is used to inflate at least one of the air chambers. In some embodiments, a non-user generated pressurized gas is used to inflate at least one of the air chambers, for example, a mechanical air pump, compressor, or compressed gas cylinder. In some preferred embodiments, the manifold is supplied using a non-user generated pressurized gas.

In some embodiments, the manifold independently controls the inflation state of each of the air chambers. In some embodiments, the inflation state of some of the air chambers is controlled together at least some of the time.

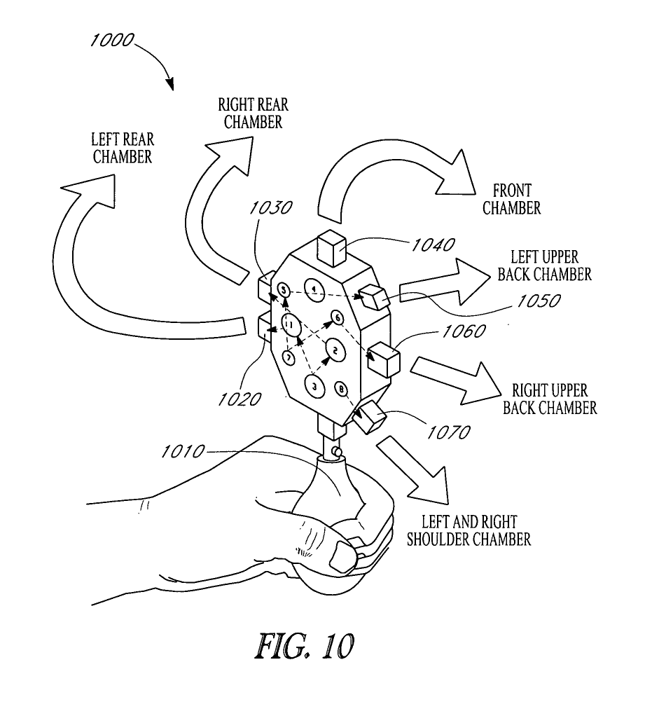

FIG. 10 illustrates an embodiment of a user controlled manifold 1000 and source of pressurized gas 1010 in fluid connection therewith suitable for use with some embodiments of the disclosed neck-and-upper-back frames and methods disclosed herein. In the illustrated embodiment, the source of pressurized gas 1010 is a hand bulb. The manifold comprises a plurality of manually activated valves of any suitable type, labeled 1-8 in FIG. 10, each of which control the inflation of one or more of the air chambers. The gas exits the manifold 1000 through a plurality of outlet ports 1020, 1030, 1040, 1050, 1060, and 1070. The correspondence between the valves, outlet ports, and air chambers for the illustrated embodiment is provided in TABLE I. In the illustrated embodiment, the shoulder air chambers are inflated together rather than separately. Those skilled in the art will understand that other arrangements for the manifold, and the control scheme are used in other embodiments.

TABLE I

| Valve | Outlet Port | Air Chamber |
|---|---|---|
| 1 | 1020 | Left Rear |
| 2 | 1030 | Right Rear |
| 3 | 1020 and 1030 | Left and Right Rear |
| 4 | 1040 | Front |
| 5 | 1050 | Left Upper Back |
| 6 | 1060 | Right Upper Back |
| 7 | 1050 and 1060 | Left and Right Upper Back |
| 8 | 1070 | Left and Right Shoulder |

Each of the disclosed neck-and-upper-back frames is also useful for implementing embodiments of the method 400 for spiral traction described above. The following description of the method references certain of the disclosed embodiments of the neck-and-upper-back frame, but those skilled in the art will understand that the methods are also applicable to other embodiments.

In step 410, the neck-and-upper-back frame 6000 is positioned and secured to the patient. In some embodiments, the shoulder 6000b and upper-back 6000c frames are first positioned and secured to the patient to provide the state illustrated in FIG. 6C. The shoulder 6000a and upper-back 6000c frames are first assembled and put on and worn by the user in much the same way that a jacket is.

The shoulder pads 6400 are positioned on the patient's shoulders. Referring to FIG. 6B, the distance between the sleeves 6446 of the rear brackets is adjusted on the upper rod 6500 of the upper-back frame 6000c according the patient's shoulder width. The lengths of the rear brackets 6430 are adjusted to match the tilt of the upper-back frame 6000c to the tilt of the lower thoracic spine. There should be a small space between the upper-back chambers 6534 and the spine. The length of the vertical rod 6510 is adjusted to the patient's waist. The chest strap 6460 and hip belt 6540 are adjusted and secured. In embodiment comprising adjustable tilting levers (7000, 8000, and 9000), the tilting levers are adjusted and locked in their rearmost positions.

The neck frame 6000a is then assembled. The lateral slats 6010 are slid through the lateral slat sleeves 6300, and the rear 6020 and front 6030 slats mounted on the lateral slats 6010 using the rear slat sleeves 6100 and front slat sleeves 6300, respectively. The occipital cups 6600 are mounted to the rear slat 6020. The chin cup 6700 is mounted to the front slat 6030.

The neck frame 6000a is mounted to the shoulder frame 6000b by engaging the bushings 6316 of the lateral slat sleeves to the heads 6352 of the tilting levers, and the retaining pins 6318 inserted. The positions of the occipital cups 6600 are adjusted such that the rear chambers engage the patient's occipital regions of the head, for example, by adjusting the forward positions of the lateral slats 6010 in the lateral slat sleeves 6300, and/or using the lower cervical tilt in embodiments with this feature, and/or adjusting the heights of the occipital cups in embodiments with this feature. TMJ spacers 300 (FIG. 6A) are inserted and positioned in the patient's mouth. The use and benefits of the TMJ spacers 300 are discussed in above. The position of the chin cup 6700 is adjusted such that the front chamber contacts the chin and extends about halfway to the angle of the mandible, for example, by adjusting the height of the chin cup in embodiments with this feature. In some embodiments, the front chamber is inflated to contact the user's chin, for example, where the chin cup is not adjustable.

In step 420, the spine is aligned. Each of the disclosed embodiments of the neck-and-upper-back frame permit the alignment of the cervical vertebrae, and at least some of the thoracic vertebrae. The disclosed devices permit orientation of the spine in any direction along the sagittal, coronal, and transverse directions prior to the application of axial traction to the spine. The following describes a preferred and non-exclusive embodiment for aligning the spine.

The neck frame permits rotational and translational positioning and alignment of the patient's head, as discussed above and illustrated in FIG. 6D. In this step, the pre-traction rotation of the neck frame is adjusted as discussed above.

The lower pre-traction tilt is then adjusted for extension or flexion as desired. In embodiments with a lower cervical tilt feature, the positions of the tilting levers 7350 (FIG. 7C) are adjusted on the shoulder pads 7400 using the push button 7358 and openings 7418 (FIG. 7B) as discussed above. In embodiments with middle cervical tilt, the tilt of the channels 9312 (FIG. 9C) is adjusted using the push button 9362 and corresponding opening 9364 of the lateral slat sleeve 9300, as discussed above. In some embodiments, the lower pre-traction tilt is adjusted by inflating the rear and/or front chambers. The side pre-traction tilt is adjusted using the rear chambers.

In step 430, traction is applied using the air chambers of the neck frame 6000a (front, right rear, and left rear). In some embodiments of this step, the patient is in different positions, for example, standing, sitting, reclining, lying down, etc.

In some embodiments, the shoulder chambers 6420 are inflated, which stretches the neck downward, thereby stretching the trapezius muscles. In some embodiments, the upper-back chambers 6534 permit user controlled flexion, extension, rotation, and lateral flexion of the upper back. For example, in some embodiments, the left or right upper-back chamber 6534 is inflated to rotate the upper back to correct rotational scoliosis. Inflating one the right or left upper-back chambers 6534 produces both rotation and lateral flexion in the thoracic spine due to the coronal orientation of the facet joints in this area of the spine. These steps are optional when the traction is repeated as discussed below.

Some embodiments comprise steps of axial distraction of the neck simultaneous with one of extension, flexion, or lateral flexion. In preferred embodiments, one of the spiral traction sequences described above (circular or figure-eight) is then applied using the air chambers of the neck frame 6000a.

In step 440, the neck frame air chambers are then deflated.

In step 450, steps 430 and 440 are optionally repeated one or more times. Repeated step(s) 430 uses the same and/or a different sequence. In some preferred embodiments, the spiral traction sequence is the figure-eight sequence, which is repeated once or twice.

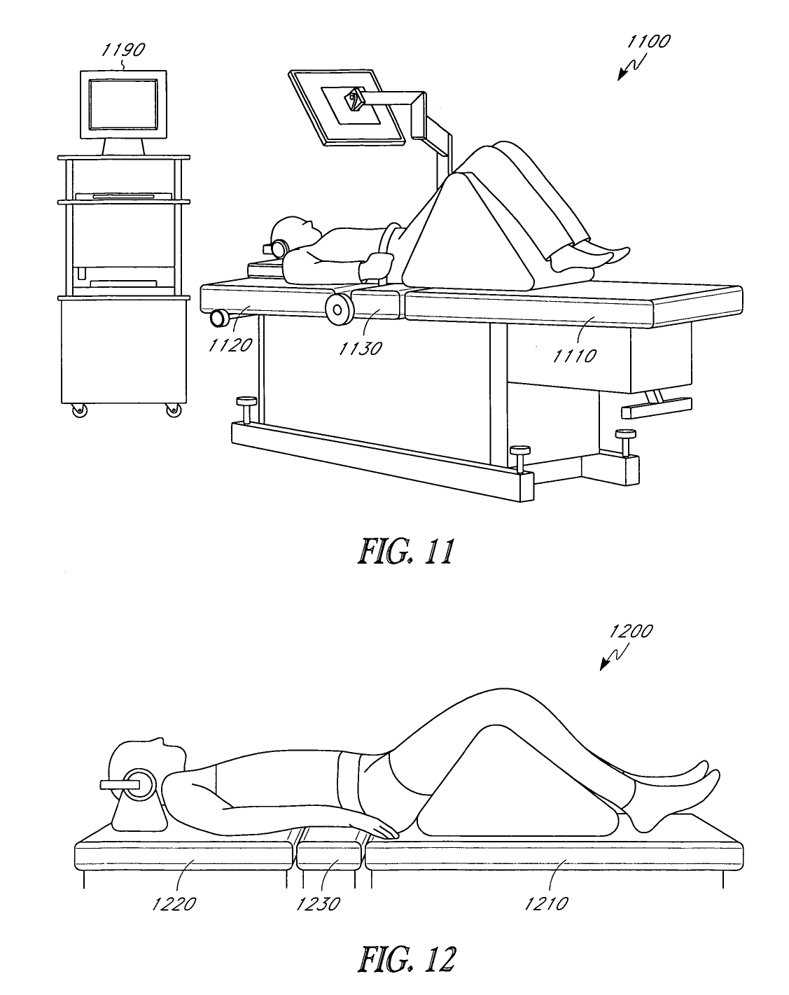

FIG. 11 shows one embodiment of a reclining chair 1100, according to an example embodiment. The reclining chair 1100 includes a top section 1120 and a bottom section 1110. The bottom section 1110 is adapted to receive a patient's bottom half or portion of the body from approximately the waist down. The top section 1120 is adapted to receive a patient's top half or portion of the body from approximately the waist up. There is a break 1130 between the top 1120 and the bottom 1110. Generally, the top section 1120 and bottom section 1110 include mechanisms for attaching a patient to the reclining chair. The top section 1120 and bottom section 1110 also include inflatable mechanisms attached to the surface near the patient and also below selected portions of either the top or bottom sections 1110, 1120 of the reclining chair 1100. The inflatable mechanisms are controllably inflated and deflated to apply spinal traction to the patient. The break 1130 between the top section 1120 and the bottom section 1110 maintains the spine and pelvis on the same plane at any given angle of tilt (flexion/extension) to the top section 1120. The break 1130 also provides flexion/extension and lateral extension of the hip joints. The reclining chair 1100 may be used with the neck and upper back frame detailed above to form a system. The reclining chair 1100 is a stationary type of reclining chair that will generally be set up in a location where it will remain for a relatively long period of time. Other embodiments of the reclining chair are portable and can be moved readily between different locales.

In one embodiment, the inflatable mechanisms are under the control of a processing device, such as a computer 1190 as shown in FIG. 11. The processing device can be hardwired to the reclining chair or may be communicatively connected to the reclining chair 1100 by way of a wireless connection. The computer 1190 is generally used to control individual portions of the inflatable mechanisms by inflating and deflating different individual portions in a sequence to move the spinal column and other portions of the reclining table 1100 to produce spiral traction. Of course, the computer 1190 may be termed as a controller which is dedicated to operating various control mechanisms associated with the reclining chair 1100, including valves for pressurized gases used to inflate various portions or valves for releasing gases from the various pressurized portions.

FIG. 12 shows one embodiment of a reclining chair 1200, according to an example embodiment. The reclining chair 1200 includes a top section 1210 and a bottom section 1220. The bottom section 1210 is adapted to receive a patient's bottom half or portion of the body from approximately the waist down. The top section 1220 is adapted to receive a patient's top half or portion of the body from approximately the waist up. There is a break 1230 between the top 1220 and the bottom 1210. Generally, the top section 1220 and bottom section 1210 include mechanisms for attaching a patient to the reclining chair 1200. The top section 1220 and bottom section 1210 also include inflatable mechanisms attached to the surface near the patient and also below selected portions of either the top or bottom sections 1210, 1220 of the reclining chair 1200. The reclining chair 1200 may be used with the neck and upper back frame detailed above. It should be noted that the break 1230 in the reclining chair 1200 is positioned at a different location when compared to the break 1130 of the reclining chair 1100. The location of the break 1230 provides an additional lateral flexion adjustment between the pelvis and the lumbar spine.

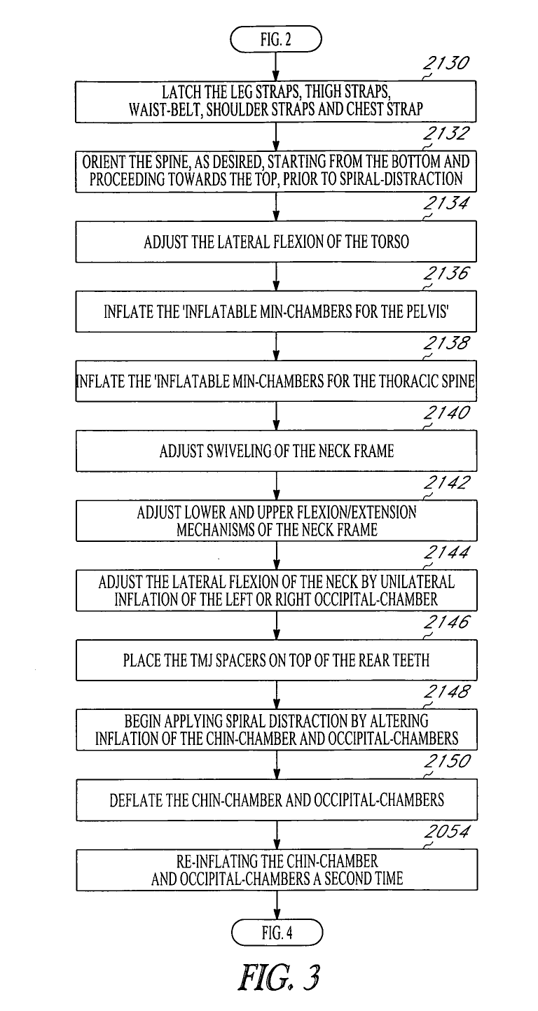

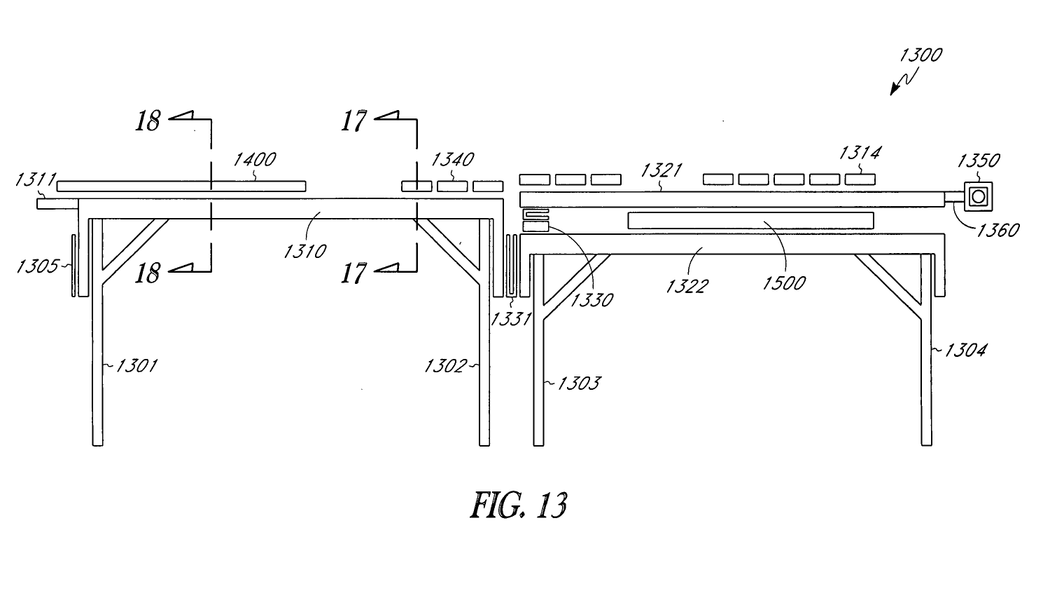

FIG. 13 is a side view of a portable reclining chair 1300, according to an example embodiment. The portable reclining chair 1300 and the stationary reclining chair, such as reclining chair 1100, have many of the same features. As shown in FIG. 13, the reclining chair 1300 includes a top section 1320 and a bottom section 1310. The bottom section 1310 is adapted to receive a patient's bottom half or portion of the body from approximately the waist down. The top section 1320 is adapted to receive a patient's top half or portion of the body from approximately the waist up. There is a break 1330 between the top 1320 and the bottom 1310. The break 1330 in the portable reclining chair includes a hinge 1331 which is positioned between the top section 1320 and the bottom section 1310 of the reclining chair 1300. Both the top section 1320 and bottom section 1310 are provided with folding legs, 1301, 1302, 1303, 1304. The folding legs may be folded and then the top portion 1320 and the bottom portion 1310 may be folded about the hinge 1331 to transport the portable reclining chair 1300. The assembly folds to form a carrying case, and the components of the cervical and upper thoracic frame (discussed with respect to FIGS. 1-10 above), which include the shoulder frame and the neck frame, that are to be used with the portable reclining chair can be stored and carried inside. Of course, the stationary types of reclining chair need not include the folding legs or the hinge between the top section 1320 and the bottom section 1310.

The bottom section 1310 includes a mechanism or mechanisms for attaching a patient to the reclining chair 1300. The bottom section 1310 also includes a thigh leg and foot support inflatable chamber 1400.

The thigh, leg and foot support inflatable chamber 1400 includes a plurality of chambers that can be inflated and deflated in various sequences. The bottom section also includes a horizontal extension 1311. The horizontal extension moves the thigh, leg and foot support horizontally to align it bellow the user's knees. Also attached to the top surface of the bottom section 1310 is a set of inflatable mini chambers 1340. The inflatable mini chambers 1340 are for pelvic orientation. The mini chambers 1340 actually are two rows of mini chambers 1340, 1340′ (shown in FIG. 17 below). The bottom section 1310 also includes a carrying handle 1305. The carrying handle 1305 allows the handler to carry the portable reclining chair 1300 when it is in its folded position. The top section 1320 also includes a first surface 1321 and a second surface 1322. The position between the first vertical surface 1321 and the second vertical surface 1322 is an inflatable chamber 1500 for reclining the torso by flexing the hip. Also included between the first vertical surface 1321 and the second vertical surface 1322 is a flexion and lateral flexion mechanism 1330. The flexion and lateral flexion mechanism 1330 includes a hinge and a rotating bushing. The flexion and lateral flexion mechanism 1330 is situated in the caudal end of the top section 1320. Also attached to the top surface 1321 are inflatable mini chambers 1314 for thoracic orientation. These mini chambers 1314 are attached between the two ends of the top surface 1321. The top surface 1321 of the top section 1320 also includes a horizontal rod 1350. The horizontal rod provides a side-to-side slidable surface for the left and right round sleeves, such as element 6446 shown in FIG. 6A, FIG. 60, FIG. 6P and FIG. 6Q (which are directly or indirectly attached to the shoulder pads). The horizontal rod 1350 is attached to a vertical bar which slides in a horizontal groove in the top surface 1321 of the top section 1320. The vertical bar allows the length of the top section to be adjusted to the height of the user. The vertical bar 1360 replaces the vertical bar of the neck and upper back frame. In addition, the horizontal rod 1350 replaces the horizontal rod of the neck and upper back frame. The center of the horizontal rod 1350 engages the vertical bar 1360 and is shaped as a square to engage a correspondingly shaped square hole in the vertical bar 1360. The square bar and the square hole or opening prevents rotation between the horizontal rod 1350 and the vertical bar. The right and left ends of the horizontal rod 1350 are rounded and positioned to engage the left and right sleeves of the shoulder pad frame and to allows side-to-side sliding of the sleeves which serves to align the shoulder frame directly over the user's shoulder. The sleeves can be attached directly to the shoulder frame, in one embodiment. In another embodiment, they can be attached to the rear bracket, with freedom of motion in various directions as described above with respect to the neck and upper bracket frame.

FIG. 14 is a side view of the inflatable chambers for the thigh and leg support 1400, according to an example embodiment. In FIG. 14, the thigh, leg and foot support inflatable chambers 1400 include three separately inflatable chambers 1401, 1402, and 1403. The three inflatable chambers or the inflatable chambers for the thigh, leg and foot support 1400 are placed on the top surface of the bottom section 1310 of the reclining chair 1300. The three inflatable chambers 1401, 1402, and 1403 inflate to support the lower extremities and specifically the thigh, leg and foot of a patient. The central chamber or chamber 1401 is inflated initially. Inflation of this chamber is generally done with the user lying supine. As the chamber 1401 is inflated, the patient's hip flexes to approximately 45 degrees and the knees are bent at approximately 90 degrees. Inflating the chamber 1401 elevates the knees and keeps the heels level with the buttocks and spine. In some applications or embodiments, the two lateral chambers 1402 and 1403 are inflated. Inflation of these chambers 1402 and 1403 with the user lying supine results in flexing of the knees and hips at 90 degrees. Inflating these lateral chambers 1402 and 1403 also elevates the feet so that the heels are level with the knees.

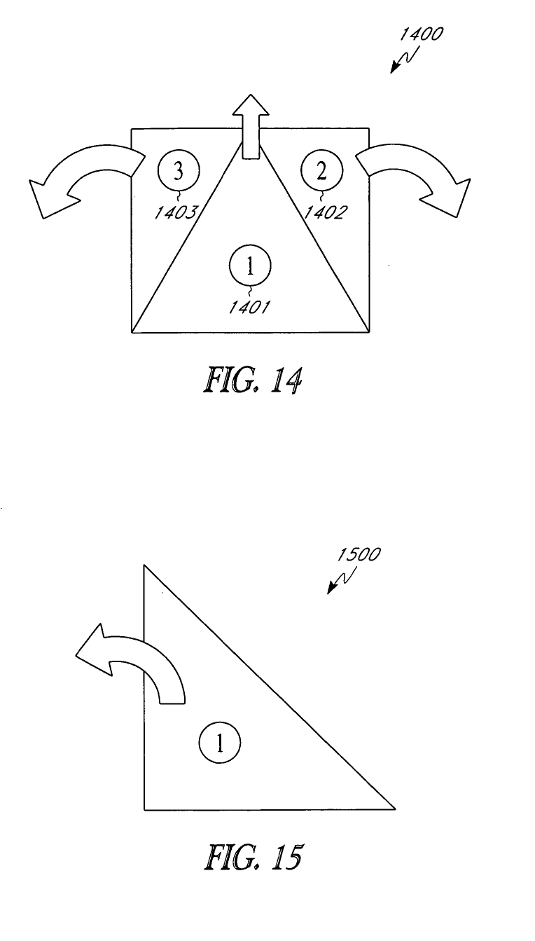

FIG. 15 is a side view of the inflatable chamber for reclining the torso by flexing the hip 1500, according to an example embodiment. The inflatable chamber for reclining the torso by flexing the hip 1500 is situated between the first surface 1321 and the second surface 1322 of the top section 1320 of the reclining chair 1300. When inflated the inflatable chamber for reclining the torso 1500 flexes the hip and maintains the top section 1321 vertical with respect to the second surface 1322. Also by changing the amount of inflation, the first surface 1321 can be placed at various angles with respect to the second surface 1322. A second surface 1322 will remain substantially horizontal while the legs 1303 and 1304 are supporting the reclining chair 1300. Thus, by selectively inflating the inflatable chamber 1500 a variety of angles can be achieved for reclining the torso and flexing the hip. As shown in FIG. 15, the inflatable chamber for reclining the torso by flexing the hip 1500 is in the shape of a right angle triangular prism. It should also be understood that the inflatable chamber 1500 could have any other shape that would recline the first surface 1321 or recline the torso with respect to the second surface 1322, such as a triangular prism, a cube or a rectangular prism.

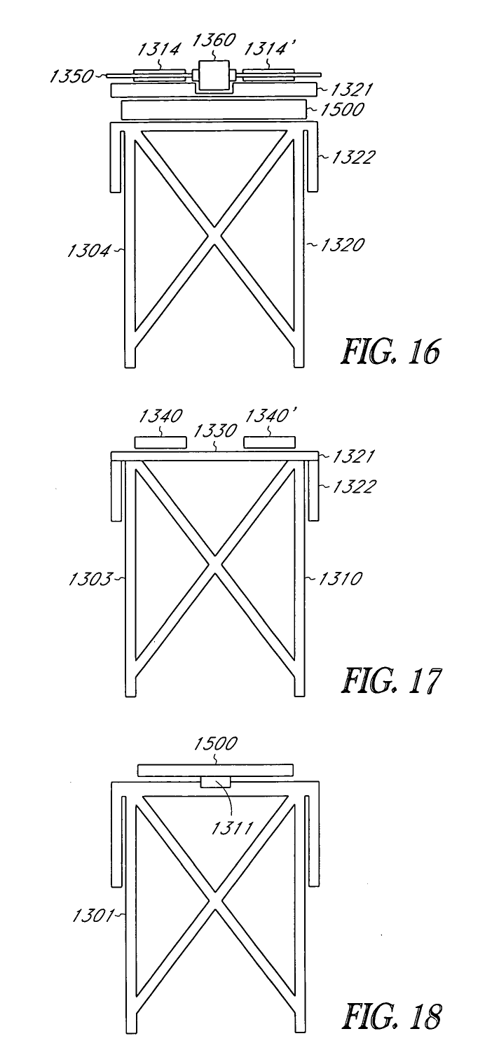

FIG. 16 is a front view of the portable reclining chair 1300, according to an example embodiment. FIG. 16 shows the inflatable chamber for reclining the torso 1500, and its orientation between the first surface 1321 and the second surface 1322. As shown, the triangularly shaped inflatable chamber 1500 has a hypotenuse that is roughly the width of the top surface 1321 of the top section 1320. The point or shorter legs of the triangle result in a point that is roughly aligned with the flexion and lateral flexion mechanism 1330 (see FIG. 13). It is also shown in FIG. 16 that the inflatable mini chambers for a thoracic orientation 1314 include a row of inflatable mini chambers 1314 on one side of the table or reclining chair 1300 and a second row along the other side of the table or reclining chair 1300. The other row is designated as 1314′. Also shown is the vertical bar 1360 and the horizontal rod 1350, which is engaged with the vertical bar 1360 as discussed above. Also shown from the front view is one of the folding legs 1304. The top surface 1321 includes a groove or channel 1323 in which the vertical bar 1360 slides.

FIG. 17 is a cross sectional view of the portable reclining chair along line 17-17 in FIG. 13, according to an example embodiment. The cross sectional view shows the inflatable mini chambers 1340, 1340′ that are oriented for pelvic orientation. One set of mini chambers 1340 is positioned on one edge of the table and another set of inflatable mini chambers 1340′ is on the other edge of the reclining chair 1300. Each row of inflatable mini chambers 1340 and 1340′ is a caudal row that engages the pelvis of the patient or user. The flexion and lateral flexion mechanism 1330 is a lockable pivoting bushing that allows the left and right lateral flexion of the lumbar spine and top section 1321 of the top portion or section 1320 of the reclining chair 1300. A hinge mechanism is on the top of the rotating bushing and allows the hips or extend. The top section hinge of the flexion and lateral flexion mechanism 1330 also allows the top section 1320 to recline up or down. In other words, the top section hinge portion of the flexion and lateral flexion mechanism 1330 allows the top surface 1321 to recline up or down with respect to the second surface 1322.

Returning briefly to FIG. 16, the inflatable mini chambers for thoracic orientation 1344, 1344′ each are a cephalic row that engages the thoracic spine. The cephalic row can slide horizontally from top to bottom in order to adjust to the height of the user. In another embodiment, there is no slider mechanism and the cephalic row cannot adjust but is stationary. In addition, a row over the lumbar spine is optional in one embodiment. The rows of mini chambers 1344, 1344′ and 1340 and 1340′ can vary in size or they can all be the same size. When the rows of mini chambers 1344 and 1344′ and 1340 and 1340′ are inflated or inflated in a particular sequence, they rotate the spine.

FIG. 18 is a cross sectional view of the portable reclining chair 1300 along line 18-18 in FIG. 13, according to an example embodiment. FIG. 18 shows the bottom section 1310 of the reclining chair 1300 which supports the buttocks, thighs, legs and feet of the patient or user. The cross section is taken through the bottom section 1310 of the reclining chair 1300. The cut line goes through the inflatable chambers 1500. As shown in FIG. 18, the inflatable chambers 1500 are actually attached to the horizontal extension 1311. The horizontal extension 1311 centers the inflatable chambers 1500 over the knee. Also shown in FIG. 18 is one of the sets of folding legs 1301.

FIGS. 13 through 18 discussed a portable reclining chair 1300. Stationary reclining chairs, such as reclining chair 1100, generally will incorporate all or some of the features of the portable reclining chair 1300. For a stationary device however, a hinge between the top and bottom sections is not needed nor is a carrying handle needed, nor or the folding legs needed. Generally the stationary model will be more substantial with additional weight, size and stability.

Belts and straps are used to secure the patient or user to a reclining chair 1100, 1200, 1300. Generally there is a waist belt, shoulder straps, chest straps and thigh straps, and leg straps. The waist belt includes left and right straps that are attached to the side of the cephalic end of the bottom section, such as bottom section 1310 in FIG. 13. A free end on one strap has a buckle that latches to the free end of the other strap. The shoulder straps generally have one strap for each of the left and right shoulder and are attached to the side of the caudal end of the top section 1320. The free ends latch or buckle to the anterior of the ipsilateral shoulder frame. The chest straps are also attached to the left and right shoulder straps, respectively. The free end on one strap has a buckle that latches to the free end of the other straps. There are two straps that are attached approximately to the mid-line of each of the left and right sides of the bottom section 1310 of the reclining chair 1300. One strap and a corresponding strap are attached to the other side of the bottom section and are wrapped over the thighs of the user. The other strap and its corresponding strap are attached to the other side of the bottom section and are wrapped over the legs. A free end on one strap has a buckle that latches to the free end of the corresponding strap and is attached to the other side of the bottom section.

In other embodiments, there may be a design variation, such as using the neck and upper back frame device with a standard reclining chair. The vertical bar of the neck and upper back frame device can be coupled to a clip that is fastened to the rest of the standard reclining chair. In some embodiments, the mechanism for flexion or lateral flexion of the torso, such as element 1330, may be eliminated. Also, there may be provided a knee rest with mechanical horizontal and vertical extension mechanisms. A separate and removable cushion or mechanical knee rest that slides horizontally and elevates vertically may be either built into or separate from the reclining chair and can replace the inflatable thigh, leg, foot support chambers 1400. There can also be provided a foot rest with mechanical horizontal and vertical extension mechanisms. This too would be a separate and removable cushion or mechanical foot rest that slides horizontally and elevates vertically and is either built into or separate from the reclining chair 1300. This could replace the inflatable thigh, leg, foot support chambers 1400.

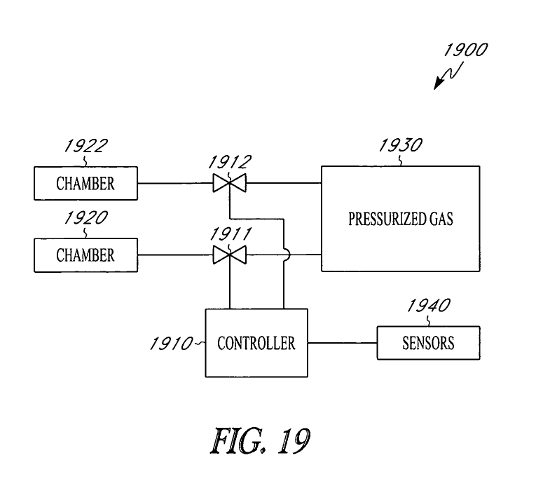

FIG. 19 is a schematic of a control mechanism 1900 for a reclining chair, according to an example embodiment. FIG. 19 shows a control mechanism 1900. The control mechanism includes a controller 1910. The controller can be a manual control or an electronic control. The control system controls chambers or the inflation parameters for various inflatable chambers 1920 and 1922. The controller controls a set of valves 1911 and 1912. The valves 1911 and 1912 allow gas from a pressurized gas source 1930 to enter into the chambers 1920 and 1922. The controller 1910 also includes a control for deflating the chambers 1920 and 1922. Of course, the controller 1910 controllably deflates the chambers 1920 and 1922. In some embodiments, the control system includes a set of sensors or at least one sensor, as depicted by reference numeral 1940. Generally, the controller 1910 controls the parameters for inflation and deflation of the various air chambers 1922 and 1920. The air chambers can be any of the air chambers either associated with the neck frame or with the reclining chair. The inflation parameters are inflated to either orientate the spine or perform a spiral joint distraction. The various parameters that can be controlled include the sequence or when the inflation takes place, the speed at which the air flow goes in and inflates the chambers, the force or control of the pressure that is built up in each of the chambers, as well as the duration or time the chambers are inflated. By controlling the duration, the amplitude of inflation is directly or indirectly controlled. As mentioned previously, the controller 1910 can be a manual type control or an electronic control. In a manual control system, the controller 1910 is anything that allows manual control over the adjustment and lockability of mechanisms that orientate the spine prior to traction and also control the inflation of the air chambers 1922 and 1920 or any of the air chambers related to either neck and upper back frame or the reclining chair. On an electronic control, the controller may be either a dedicated microcontroller or a computer. The electronically controlled method includes but is not limited to motorized magnetic and pneumatic mechanisms that lock and orientate the spine prior to traction, electronically controlling the inflation of the air chambers 1920, 1922. The electronic controls can also control the source of pressurized gas. In addition, if a computer or microprocessor is used the controls can be any kind of programmable sequence, including a preprogrammed sequence of instructions provided by a manufacturer, an automatically programmable set of instructions which rely on input or reading from various sensors 1940 that are installed on the device and measure a variety of parameters including pressure, heat and the like. An electronic controller could also be reprogrammed by a user or a doctor or via a new instruction set from the manufacturer. In one embodiment of the invention, a remote hand-held device can be used to control the functions. The remote or hand-held device can include a display that shows the control options that are available and the choices selected. In one example, after inflation parameters for the sequence, speed, amplitude and duration are selected on the screen and could display a simulation of the spine being moved according to the selected parameters in order to help the user visualize and further modify the selection.

The sensors 1940 can be mounted on any part of the frame, such as inside the chin cup or the occipital cups of the neck frame. The sensors 1940 can be used to monitor pressure and heat and other physiological parameters of the patient. The sensors can also be used to reevaluate the options for the sequence, speed, force and amplitude and the duration of joint distraction and soft tissue mobilization. Resetting the various sensor parameters can be done manually or it can be automated.

Adjunct therapies can be performed simultaneously with distraction and mobilization and it also can be mounted on any part of the frame. Examples of adjunct therapies include laser and infra red treatments, as well as cryotherapy. It should be noted that there is no limitation to the type of adjunct therapy that can be implemented along with the distraction and mobilization provided by the neck frame and the reclining chair system.

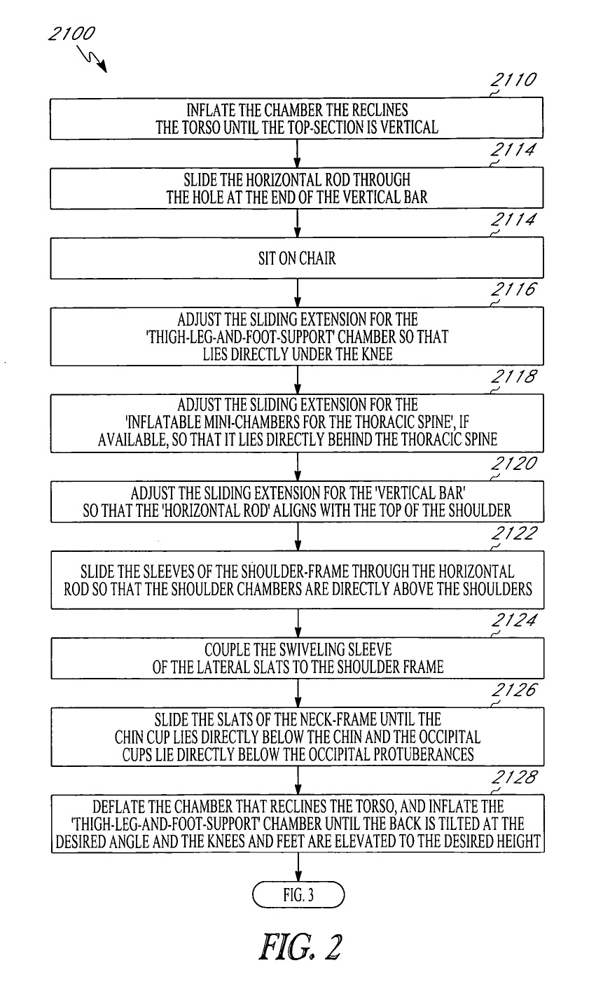

FIGS. 2-4 are a flow diagram of a method 2100 of using the system that includes the neck and upper back frame and a reclining chair, according to an example embodiment. The method 2100 includes inflating the chamber that reclines the torso until the top section is vertical 2110 and sliding the horizontal rod through the opening at the end of the vertical bar 2112. The user then sits on the reclining chair 2114. The sliding extension for the thigh, leg and foot support inflatable chamber is adjusted so that it lies directly under the knee, as depicted by reference numeral 2116. The sliding extension for the inflatable mini chambers for the thoracic spine is adjusted so that it lies directly behind the thoracic supine, as depicted by reference numeral 2118. Sliding extension for the vertical bar is adjusted so that the horizontal rod aligns with the top of the shoulder, as depicted by reference numeral 2120. The sleeves of the shoulder frame are slid through the horizontal rod so that the shoulder chambers are directly above the shoulders, as depicted by reference numeral 2122. The swiveling sleeve of the lateral slats is coupled to the shoulder frame, as depicted by reference numeral 2124. The slats of the neck frame are slid into a place until the chin cup lies directly below the chin and the occipital cups lie directly below the occipital protuberances, as depicted by reference numeral 2126. The chamber that reclines the torso is deflated and the thigh, leg and foot support chamber is inflated until the back is tilted at a desired angle and the knees and feet are elevated to a desired height, as depicted by reference numeral 2128. Leg straps, thigh straps, waist belt, shoulder straps and chest straps are then latched, as depicted by reference numeral 2130. The shoulder chambers are inflated, as depicted by reference numeral 2058. Inflating both the shoulder chambers lowers and reverses rounding of the shoulders. The spine is then oriented as desired, in flexion, extension, rotation or lateral flexion, starting from the bottom and proceeding towards the top prior to spiral distraction, as depicted by reference numeral 2132. Lateral flexion of the torso is then adjusted, as depicted by reference numeral 2134. The inflatable mini chambers for the pelvis are inflated, as depicted by reference numeral 2136. Also inflated are the inflatable mini chambers for the thoracic spine, as depicted by reference numeral 2138. The neck frame is then orientated through several adjusting steps. The swivel of the neck frame is first adjusted, as depicted by reference numeral 2140. The lower and upper flexion/extension mechanism of the neck frame is then adjusted, as depicted by reference numeral 2142. The lateral flexion of the neck by unilateral inflation of the left or right occipital chamber is then adjusted, as depicted by reference numeral 2144. Those adjustment steps orient the neck frame. The TMJ spacers are then placed on top of the rear teeth, as depicted by reference numeral 2146. Spiral distraction is then started by alternatingly inflating the chin chamber and the occipital chambers, as depicted by reference numeral 2148. These chambers are then deflated, as depicted by reference numeral 2150. The chin chamber and the occipital chamber are then reinflated, as depicted by reference numeral 2154 and this deflating and reinflating of these chambers is continued for as many times as desired, as depicted by reference numeral 2056. The left rear chamber can be inflated to stretch the left side of the neck. The right rear chamber can then be inflated to stretch the right side of the neck. Both rear chambers can be inflated to stretch the back of the neck and then front chamber can be inflated to stretch the front of the neck. The neck assembly chambers can then deflated, as depicted by reference numeral 2056 and the inflation and deflation of the neck assembly chambers can be repeated.

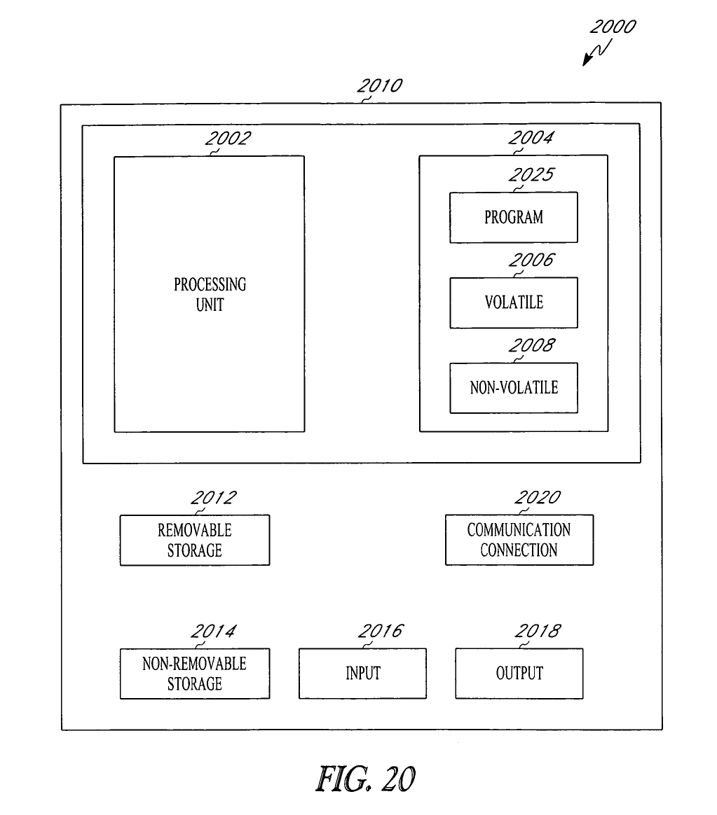

FIG. 20 is a block diagram of a computer system 2000 that executes programming for performing methods discussed herein, according to an example embodiment. A general computing device in the form of a computer 2010, may include a processing unit 2002, memory 2004, removable storage 2012, and non-removable storage 2014. Memory 2004 may include volatile memory 2006 and non-volatile memory 2008. Computer 2010 may include, or have access to a computing environment that includes, a variety of computer-readable media, such as volatile memory 2006 and non-volatile memory 2008, removable storage 2012 and non-removable storage 2014. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions. Computer 2010 may include or have access to a computing environment that includes input 2016, output 2018, and a communication connection 2020. One of the inputs could be a keyboard, a mouse, or other selection device. The communication connection 2020 can also include a graphical user interface, such as a display. The computer may operate in a networked environment using a communication connection to connect to one or more remote computers. The remote computer may include a personal computer (PC), server, router, network PC, a peer device or other common network node, or the like. The communication connection may include a Local Area Network (LAN), a Wide Area Network (WAN) or other networks.

Computer-readable instructions stored on a computer-readable medium are executable by the processing unit 2002 of the computer 2010. A hard drive, CD-ROM, and RAM are some examples of articles including a computer-readable medium. For example, a computer program 2025 capable of providing a generic technique to perform access control check for data access and/or for doing an operation on one of the servers in a component object model (COM) based system according to the teachings of the present invention may be included on a CD-ROM and loaded from the CD-ROM to a hard drive. The computer-readable instructions allow computer system 2000 to provide generic access controls in a COM based computer network system having multiple users and servers.

A machine-readable medium, such as discussed above, provides instructions that, when executed by a machine, cause the machine to perform specific sequences of inflating and deflating the various gas chambers of the system. This includes controlling the timing, duration, sequence, force and pressure of the various inflatable portions of the above described system. The machine readable medium may also include instruction sets for carrying out various instructions in response to inputs for the sensor or sensors (see 1940 of FIG. 19). Furthermore, the machine readable medium may also include instruction sets for carrying out some portions of the method or methods discussed herein, such as method 2000.

Orient the spine in any direction, prior to traction, by separately controlling each of the three directions of motion of the Spine—Flexion/Extension, Lateral Flexion (left and right) and Rotation (left and right). Then distract Spinal Joints in a “Spiral-Pathway” by alternatingly tilting the head left, right, forward and back (Left/Right Lateral Flexion, Flexion, and Extension—in any sequence), while simultaneously lifting the head and elongating the spine. For example, one possible inflation sequence involves lifting the left rear chamber, both rear chambers, right rear chamber and the front chamber; then deflating these chambers and repeating the procedure at least once.

The portable or stationary bench or a reclining-chair allows the patient to be sitting upright, reclined or laying supine when the spiral traction methods are applied. The portable or stationary bench or a reclining-chair anchors and stabilizes the lower body at the ankles, thighs and waist and orients the hip, and hence the lower spine, in flexion and lateral flexion. The portable or stationary bench or a reclining-chair also includes inflatable air chambers on the resting surface that flex and rotate the thoracic and lumbar spine. Once the spine is oriented and tension is created at the point of desired release, the alternating inflation of the inflatable chambers situated bellow the chin and the occipital protuberances produce ‘spiral’ joint-distraction and soft-tissue mobilization through the spine, beginning from the top of the spine and gradually proceeding towards the bottom of the spine. Also disclosed are control methods and sensors that can be controlled manually or by way of a set of instructions associated with a media. The portable or stationary bench or a reclining-chair is communicatively coupled to a computer, processor or microprocessor which acts in response to a set of instructions to carry out various manipulation procedures. The set of instructions can be pre-programmable, re-programmable, or auto-programmable. The controls may be used to control the force, speed and amplitude of inflation for each individual chamber. The system may also be provided with sensors that interface with the control system and provide feedback to the control system.

When the reclining chair is used in combination with the neck-and-upper-back frame, both devices act together can provide a spiral spinal-joint mobilization, or manipulation, for the entire spine. The spiral spinal-joint mobilization, or manipulation results in full-spine spinal-joint distraction and decompression, and simultaneous multi-directional paraspinal soft-tissue mobilization and release.

Spiral spinal-joint decompression and soft-tissue release begins at the top of the spine, in the sub-occipital area, by the action of the inflatable chambers situated bellow the left and right occipital protuberances and the chin. The ‘spiral-path’ of release and decompression of spinal-joints and soft-tissues gradually and incrementally progresses down to the lower segments of the spine.

The foregoing description of the specific embodiments reveals the general nature of the invention sufficiently that others can, by applying current knowledge, readily modify and/or adapt it for various applications without departing from the generic concept, and therefore such adaptations and modifications are intended to be comprehended within the meaning and range of equivalents of the disclosed embodiments.

It is to be understood that the phraseology or terminology employed herein is for the purpose of description and not of limitation. Accordingly, the invention is intended to embrace all such alternatives, modifications, equivalents and variations as fall within the spirit and broad scope of the appended claims.

1. A system for spinal traction comprising:
a neck and upper back frame further comprising:
an extensible chin chamber sized and dimensioned to engage a user's chin; and
an extensible occipital region chamber sized and dimensioned to engage an occipital region of a user's head; and
a reclining chair further comprising:
a top section adapted to engage the user's spine, the top section including;
a first row of extensible thoracic chambers for engaging at least a portion of the user's spine; and
a second row of extensible thoracic chambers for engaging at least a portion of the user's spine; and
bottom section adapted to support the buttocks, thighs, feet and legs; and
a control mechanism for extending and retracting the chin chamber, the occipital chamber, the first row of thoracic chambers and the second row of thoracic chambers.

2. The system of claim 1 further comprising an extensible chamber for tilting a top surface of the top section with respect to a top surface of the bottom section.

3. The system of claim 1, wherein the top section further comprises:
a first set of extensible lumbar chambers for engaging at least a portion of the user's spine; and
a second set of extensible lumbar chambers for engaging at least a portion of the user's spine.

4. The system of claim 1, wherein the bottom section further comprises:
a first set of extensible pelvic chambers for engaging the user's pelvis.

5. The system of claim 1, wherein the bottom section includes a thigh leg foot pad.

6. The system of claim 1, wherein the bottom section includes a thigh leg foot extensible chamber.

7. The system of claim 1, wherein the control mechanism is configured to extend at least one of the chin chamber, the occipital region chamber, the first row of extensible thoracic chambers and the second row of extensible thoracic chambers in a desired sequence.

8. The system of claim 7 wherein the control mechanism comprises an electronic control system.

9. The system of claim 7 wherein the control mechanism is operably coupled to a processor.

10. The system of claim 9 further comprising a machine-readable medium that provides instructions that, when executed by the processor, cause extension of at least one of the chin chamber, the occipital region chamber, the first row of extensible thoracic chambers and the second row of extensible thoracic chambers in a desired sequence.

11. The system of claim 7, wherein the control mechanism extends and retracts the various chambers in a desired sequence to place forces on the body resulting in spiral traction.

12. A method comprising:
fluidly coupling a set of extensible chambers on a neck and upper back frame to a source of pressurized fluid;
fluidly coupling a set of extensible chambers on a reclining table to a source of pressurized fluid; and
controllably extending and retracting the extensible chambers associated with the neck and upper back frame, and the extensible chambers associated with the reclining table in a desired sequence.

13. The method of claim 12 wherein controllably extending and retracting the extensible chambers associated with the neck and upper back frame, and the extensible chambers associated with the reclining table in a desired sequence comprises generating spirally acting forces along the extensible chambers.

14. The method of claim 12, further comprising attaching the neck and upper back frame to a patient.

15. The method of claim 12, further comprising orienting a patient relative to the reclining table.

16. The method of claim 15, further comprising strapping the patient to the reclining table.

17. The method of claim 12, further comprising tilting one portion of the reclining table with respect to another portion of the reclining table.

18. The method of claim 17 wherein tilting one portion of the reclining table with respect to another portion of the reclining table is accomplished by extending at least one chamber.

19. A system for spinal traction comprising:
a frame configured to releasably engage a neck and back portions of a user that includes extensible members configured to independently apply forces to at least an occipital region and a mandibular region of the user;
a reclining chair configured to be coupled to the frame, the reclining chair comprising:
a first section structured to receive at least a portion of a spinal region of the user that further includes extensible members positioned on the first section and configured to engage at least a portion of the spine of the user; and
a second section coupled to the first section and structured to receive a lower region of the user.

20. The system of claim 19, wherein the extensible members are fluidly sealed members, further comprising a control mechanism configured to selectively provide a pressurized fluid to the fluidly sealed members.

21. The system of claim 19, wherein the first section comprises at least one row of extensible mini-chambers positioned on a surface of the first section and extending laterally across the surface of the first section that are configured to engage thoracic and lumbar regions of the user.

22. The system of claim 19, wherein the second section comprises at least one row of extensible mini-chambers positioned on a surface of the second section and extending laterally across the surface of the second section that are configured to engage a pelvic region of the user.

23. The system of claim 19, wherein the second section comprises at least one extensible chamber positioned on the surface of the second section and adjacent the at least one row of extensible mini-chambers and configured to engage the thigh, leg and foot portions of the user.

24. The system of claim 19, wherein the first section is positionable relative to the second section along an axis longitudinal to the first section and the second section to provide lateral flexion to the user.

25. The system of claim 19, wherein the first section comprises an extensible member that underlies the surface of the first section that is configured to flex and extend the first section relative to the second section about a hinge positioned along the transverse axis of the table.

26. The system of claim 23, wherein the second section comprises an extension that is longitudinally translatable outwardly from the second section to align the at least one extensible chamber relative to a knee portion of the user.

27. The system of claim 19, wherein the first section comprises at least one restraint operable to retain an upper body portion of the user to the first section.

28. The system of claim 19, wherein the second section comprises at least one restraint operable to retain a lower body portion of the user to the second section.

29. The system of claim 20, wherein the control mechanism comprises a pressurized fluid source fluidly coupled to the extensible members to define a fluid flow path, wherein the control mechanism further comprises at least one valve interposed in the fluid flow path.

30. The system of claim 29, wherein the control mechanism comprises an electronic control system that is operable to control at least one of an inflation sequence, an inflation speed, an inflation pressure and an inflation duration.

31. The system of claim 3, wherein the control mechanism is configured to extend at least one of the first row of extensible lumbar chambers and the second row of extensible lumbar chambers in a desired sequence.

32. The system of claim 4, wherein the control mechanism is configured to extend at least one of the first row of extensible pelvic chambers and the second row of extensible pelvic chambers in a desired sequence.

33. The system of claim 31, wherein the control mechanism comprises an electronic control system.

34. The system of claim 32, wherein the control mechanism comprises an electronic control system.

35. The system of claim 31, wherein the control system is operably coupled to a processor.

36. The system of claim 32, wherein the control system is operably coupled to a processor.

37. The system of claim 35, comprising a machine readable medium that provides instructions that, when executed by the processor, cause expansion of at least one of the first row of extensible lumbar chambers and the second row of extensible lumbar chambers in a desired sequence.

38. The system of claim 36, comprising a machine readable medium that provides instructions that, when executed by the processor, cause expansion of at least one of the first row of extensible pelvic chambers and the second row of extensible pelvic chambers in a desired sequence.

39. The system of claim 31, wherein the control mechanism extends and retracts the various chambers in a desired sequence to place forces on the user resulting in spiral traction.

40. The system of claim 32, wherein the control mechanism extends and retracts the various chambers in a desired sequence to place forces on the user resulting in spiral traction.
